(19) United States
(12) Patent Application Publication
(10) Pub. No.: US 2025/0287968 A1
(43) Pub. Date: Sep. 18, 2025

(54) COMPOSITION FOR CLOTTING MILK, METHODS AND USES THEREOF

(71) Applicant: Chr. Hansen A/S, Hoersholm (DK)

(72) Inventors: Sebastien Roustel, Hoersholm (DK); Johannes Maarten Van den Brink, Hoersholm (DK); Surender Kumar Dhayal, Hoersholm (DK); Martin Lund, Hoersholm (DK)

(73) Assignee: Chr. Hansen A/S, Hoersholm (DK)

(21) Appl. No.: 18/580,350

(22) PCT Filed: Jul. 19, 2022
(86) PCT No.: PCT/EP2022/070216

§ 371 Date: Jan. 18, 2024

(30) Foreign Application Priority Data
Jul. 20, 2021 (EP) 21186598.5

(51) Int. Cl. A23C 19/04

(52) U.S. Cl. CPC A23C 19/04; A23C 2220/106

(57) ABSTRACT

This invention describes a composition for clotting milk wherein the composition contributes simultaneously to the increase of yield of cheese curd and to the maintenance of adequate proteolysis values which are responsible for the proper development of texture and flavor in specific types of cheese.

### REFERENCE TO A SEQUENCE LISTING

This application contains a Sequence Listing in computer readable form which is incorporated herein by reference. The contents of the electronic sequence listing was created on May 28, 2024, named SQ.xml and has 62,774 bytes in size. This replaces the previously filed sequence listing.

### FIELD OF THE INVENTION

This invention describes a composition for clotting milk wherein the composition contributes simultaneously to the increase of yield of cheese curd and to the maintenance of adequate proteolysis values which are responsible for the proper development of texture and flavor in specific types of cheese or for specific cheese markets.

The composition herein disclosed may comprise at least two different coagulants, preferably having different clotting to proteolysis (C/P) ratios, wherein said coagulants are added, in a sequential order or simultaneously, for clotting the milk used for making cheese. The combination of the coagulants minimizes the loss of fat particles and fine curd particles into the whey, thereby increasing the yield of cheese curd, while simultaneously maintaining or improving proteolysis levels such that the development of texture and flavor still occurs.

This invention also relates to a method to obtain higher cheese yield and proper proteolysis by using the composition herein disclosed.

### BACKGROUND OF THE INVENTION

Aspartic proteases (EC 3.4.23) are proteolytic enzymes, preferably peptidases, more preferably endopeptidases, having two aspartic acid residues in the active site that are relevant for their catalytic activity. These enzymes can be found, for example, in animals, plants or fungi and are relevant in the cheese-making industry as milk-clotting enzymes.

Enzymatic coagulation of milk by milk clotting enzymes or coagulants is one of the most important processes in the manufacture of cheeses. The enzymatic coagulation of milk by milk clotting enzymes or coagulants can be made by chymosins (EC 3.4.23.4), pepsins (EC 3.4.23.1), endothiapepsins (EC 3.4.23.22) or mucorpepsins (EC 3.4.23.23). For the sake of completeness, a chymosin is to be considered as a coagulant, however, not all coagulants are chymosins.

Enzymatic milk coagulation is a two-phase process: a first phase where a proteolytic enzyme attacks K-casein, resulting in a metastable state of the casein micelle structure and a second phase, where the milk subsequently coagulates and forms a coagulum.

Chymosin (EC 3.4.23.4) and pepsin (EC 3.4.23.1) are milk clotting enzymes of the mammalian stomach. Endothiapepsin (EC 3.4.23.22) is a microbial milk clotting enzyme from Cryphonectria parasitica. Mucorpepsin (EC 3.4.23.23) is a microbial milk clotting enzyme from Rhizomucor (pusillus or miehei).

When produced in the gastric mucosal cells, chymosin and pepsin occur as enzymatically inactive pre-prochymosin and pre-pepsinogen, respectively. When chymosin is excreted, an N-terminal peptide fragment, the pre-fragment (signal peptide) is cleaved off to give prochymosin including a pro-fragment. Prochymosin is a substantially inactive form of the enzyme which, however, becomes activated under acidic conditions to the active chymosin by autocatalytic removal of the pro-fragment. This activation occurs in vivo in the gastric lumen under appropriate pH conditions or in vitro under acidic conditions.

Chymosin has a high specificity and predominantly clots milk by cleavage of a single 104-Ser-Phe-|-Met-Ala-107 bond in kappa-chain of casein. As a side-activity, chymosin also cleaves alpha-casein primarily between Phe23 and Phe24 and beta-casein primarily between Leu192 and Tyr193. The resulting peptides alphaS1 (1-23) and beta (193-209) are further degraded by proteases and peptidases from microbial cultures added to the ripening cheese. An alternative name used in the art is rennin.

Chymosins (EC 3.4.23.4) are marketed under the trade names Chymostar™ by DuPont, Maxiren® by DSM, CHY-MAX®, CHY-MAX M®, CHY-MAX® Supreme by Chr. Hansen. Chymosins (EC 3.4.23.4) are known as high C/P coagulants or high C/P ratio coagulants.

Endothiapepsin clots milk by catalyzing the hydrolysis of peptide bonds through the action of its two aspartate residues. This enzyme is marketed under the trade name Suparen® by DSM, THERMOLASE® by Chr. Hansen. Endothiapepsin is known as a low C/P coagulant or low C/P ratio coagulant.

Mucorpepsin is marketed under the trade name of HANNILASER or HANNILASER/MICROLANT® both by Chr. Hansen, Fromase® by DSM, Marzyme® by DuPont.

Kim et al 2004 relates to a combination of a chymosin and Cryphonectria parasitica protease for manufacturing Cheddar cheese where a chymosin-to-Cryphonectria parasitica ratio between 0:1 to 67:33 was found suitable to independently control Cheddar cheese meltability and hardness without a significant level of bitterness. Kim et al 2004 is, however, silent about increasing yield in cheese and how to promote or achieve the stability of the above-mentioned combination of chymosin and protease. Additionally, the ratio of Cryphonectria parasitica protease seems to be significantly higher than the one of the present invention.

### WO2002036752

WO2002036752 relates to a non-bovine milk-clotting enzyme, in particular a milk-clotting enzyme derived from camel. Further, this patent document discloses milk clotting compositions comprising a bovine milk clotting enzyme selected from prochymosin, chymosin and pepsin and a non-bovine milk clotting enzyme selected from prochymosin, chymosin, pepsin and a microbial aspartic protease selected from M. pusillus or M. miehei; and a method of manufacturing cheese from milk, comprising adding to milk a milk clotting effective amount of such a composition.

In another embodiment, WO2002036752 discloses a milk clotting composition comprising a milk clotting bovine enzyme selected from prochymosin, chymosin and pepsin and a non-bovine milk clotting enzyme selected from prochymosin, chymosin and pepsin including such a composition where the milk clotting activity ratio between the bovine and the non-bovine milk clotting enzyme is in the range of 1:99 to 99:1, including a composition where at least 2% of the milk clotting activity is from the non-bovine enzyme such as at least 5%, 10%, 20%, 50%, 75%, 90% or 98% of the activity. In one preferred embodiment, the non-bovine enzyme in such a mixed composition is derived from Camelus dromedarius.

Finally, WO2002036752 discloses that a camel chymosin gives lower dry matter loss to whey, reflecting an expectation of a higher cheese yield.

### WO2016128476

WO2016128476 discloses a milk clotting composition comprising a camel chymosin and a further milk clotting enzyme, which can be a bovine chymosin (Examples 4, 5) or a mucorpepsin (Example 4). The purpose of WO2016128476 is to obtain a composition providing high C/P ratio, which contributes to a higher cheese yield, and simultaneously reducing firmness of the cheese, such that a softer cheese is obtained.

In conclusion, WO2002036752 and WO2016128476 are silent about compositions comprising a microbial aspartic protease from Cryphonectria parasitica and a further milk-clotting enzyme. Further, the available prior art is silent about compositions contributing to the increase of yield in cheese curd while simultaneously maintaining proper levels of proteolysis for cheese making. So far, the prior art have been focused on improving the yield in cheese curd for cheese segments wherein the maintenance of texture and/or flavor may be of less relevance and the cheese producer and consumer were not concerned with having less proteolysis. Finally, the available prior art is silent about formulations that promote the stability of the above-mentioned compositions.

### SUMMARY OF THE INVENTION

Coagulants with a low C/P ratio have shown to contribute to the development of flavor and/or texture in cheese by promoting faster proteolysis over time. As a consequence of the proteolysis, these coagulants contribute poorly to cheese yield. In contrast, coagulants with a high C/P ratio have shown to improve yields in different cheese segments, such as Pasta-Filata, Continental, Cheddar, Queso Fresco, leading to less proteolysis over time due to a high C/P ratio of the coagulant.

However, in some specific cheese markets there is a need to combine some degree of proteolysis for enhancing flavor formation and/or a specific desired texture with an increase in yield of cheese curd. This cannot be achieved with a high C/P ratio coagulant as only the need of increased yield in cheese curd would be obtained, neither can it be achieved with a low C/P ratio coagulant as only the need of increasing proteolysis would be accomplished. Thus, in some specific cheese markets having only low C/P ratio coagulant or only high C/P ratio coagulant may not be, in fact, desirable. For these markets what is desired is to have one coagulant that simultaneously is responsible for high cheese yield and proper flavor and/or texture formation, or in alternative a combination of coagulants that together lead to high cheese yield and proper flavor and/or texture formation.

This invention shows that a combination of different coagulants, for example, the combination of any low C/P ratio coagulant with any high C/P ratio coagulant does not necessary solve the need of having increased yield in cheese curd with a proper proteolysis levels.

### First Aspect of the Invention

An aspect of this invention shows that specific combinations of a low C/P ratio coagulant and a high C/P ratio coagulant are able to solve the above-mentioned problem of having a proper proteolysis over time while simultaneously contributing to an adequate cheese yield, provided the low C/P ratio coagulant is derived from Cryphonectria.

The solution to the above problem is a composition comprising a low C/P ratio coagulant as a first coagulant, wherein the first coagulant is derived from Cryphonectria, such as Cryphonectria parasitica, and high C/P ratio coagulant as a second coagulant.

Preferably the solution to the above problem is a composition for clotting milk comprising:
a first coagulant derived from or of Cryphonectria, preferably Cryphonectria parasitica; and
a second coagulant derived from or of Camelus, preferably Camelus dromedarius or Camelus bactrianus, more preferably Camelus dromedaries, or a second coagulant derived from or of Bos, preferably Bos taurus.

In a preferred embodiment, the first coagulant derived from or of Cryphonectria, preferably Cryphonectria parasitica may be an endothiapepsin.

In a preferred embodiment, the first coagulant derived from or of Cryphonectria, preferably Cryphonectria parasitica, may have at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2, more preferably wherein the first coagulant is SEQ ID NO: 1 or 2 or a genetically-engineered coagulant derived from SEQ ID NO: 1 or 2. In another preferred embodiment, the genetically-engineered coagulant derived from SEQ ID NO: 1 or 2 has at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2.

In a preferred embodiment, the first coagulant derived from or of Cryphonectria, preferably Cryphonectria parasitica, may be an encapsulated coagulant.

In a preferred embodiment, the first coagulant derived from or of Cryphonectria, preferably Cryphonectria parasitica, may be an endothiapepsin or an encapsulated endothiapepsin.

In a preferred embodiment, the second coagulant derived from or of Camelus may have at least 80% sequence identity with SEQ ID NO: 3 or 4 or 5, preferably at least 85% or at least 90% or at least 91% or at least 92% or at least 93% or at least 94% or at least 95% or at least 96% or at least 97% or at least 98% or at least 99% sequence identity with SEQ ID NO: 3 or 4 or 5, more preferably wherein the second coagulant is SEQ ID NO: 3 or 4 or 5, or a genetically-engineered coagulant derived from SEQ ID NO: 3 or 4 or 5. In a more preferred embodiment, the second coagulant derived from or of Camelus or a genetically-engineered coagulant derived from SEQ ID NO: 3 or 4 or 5 may be any of the sequences SEQ ID NO: 8-52. Any of these sequences may be equally chosen, in particular as these sequences correspond to high C/P ratio coagulants, in particular derived from or of camel.

In a preferred embodiment, the second coagulant derived from or of Bos may have at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7, more preferably wherein the second coagulant is SEQ ID NO: 6 or 7, or a genetically-engineered coagulant derived from or of SEQ ID NO: 6 or 7. In another preferred embodiment, the genetically-engineered coagulant derived from or of SEQ ID NO: 6 or 7 has at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7; or a genetically-engineered coagulant of SEQ ID NO: 6 or 7.

More preferably, the solution to the above problem is a composition for clotting milk comprising a first coagulant having at least 80% sequence identity with SEQ ID NO: 1 or 2 and a second coagulant having at least 80% sequence identity with SEQ ID NO: 3 or 4 or 5 or 6 or 7. Preferably, the first coagulant may have at least 85% or at least 90% or at least 91% or at least 92% or at least 93% or at least 94% or at least 95% or at least 96% or at least 97% or at least 98% or at least 99% or 100% sequence identity with SEQ ID NO: 1 or 2, or a genetically-engineered coagulant derived from SEQ ID NO: 1 or 2, which may have at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2. Preferably, the second coagulant may have at least 85% or at least 90% or at least 91% or at least 92% or at least 93% or at least 94% or at least 95% or at least 96% or at least 97% or at least 98% or at least 99% or 100% sequence identity with SEQ ID NO: 3 or 4 or 5, or a genetically-engineered coagulant derived from SEQ ID NO: 3 or 4 or 5. The genetically-engineered coagulant derived from SEQ ID NO: 3 or 4 or 5 may be any of the sequences SEQ ID NO: 8-52. Any of these sequences may be equally chosen, in particular as these sequences correspond to high C/P ratio coagulants, in particular derived from or of camel. Alternatively, the second coagulant may have at least 85% or at least 90% or at least 91% or at least 92% or at least 93% or at least 94% or at least 95% or at least 96% or at least 97% or at least 98% or at least 99% or 100% sequence identity with SEQ ID NO: 6 or 7, or a genetically-engineered coagulant derived from or of SEQ ID NO: 6 or 7, which may have at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7.

In a preferred embodiment, the composition now disclosed may be a liquid composition or a powder composition.

In a preferred embodiment, the composition or compositions now disclosed may comprise at most 25% of the first coagulant derived from or of Cryphonectria parasitica, preferably at most 23%, more preferably at most 15%, even more preferably at most 6%; wherein the % indicates the IMCU/ml of the first coagulant relative to the IMCU/ml of total coagulants in the composition. Preferably, the composition now disclosed may comprise 1-25% or 1-23% or 1-20% or 1-15% or 1-14% of the first coagulant derived from or of Cryphonectria parasitica, more preferably 1-10% or 1-6% of the first coagulant derived from or of Cryphonectria parasitica, even more preferably 2-5% of the first coagulant derived from or of Cryphonectria parasitica or 1-2% of the first coagulant derived from or of Cryphonectria parasitica, wherein the % indicates the IMCU/ml of the first coagulant relative to the IMCU/ml of total coagulants in the composition. Preferably, the first coagulant derived from or of Cryphonectria parasitica may have at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2.

In a preferred embodiment, the composition or compositions now disclosed may at least 75% of the second coagulant derived from or of Camelus dromedarius or Camelus bactrianus or Bos taurus, preferably at least 77%, more preferably at least 85%, even more preferably at least 94%; wherein the % indicates the IMCU/ml of the second coagulant relative to the IMCU/ml of total coagulants in the composition. Preferably, the composition now disclosed may comprise 75-99% or 77-99% or 80-99% or 85-99% or 86-99% of the second coagulant derived from or of Camelus dromedarius or Camelus bactrianus or Bos taurus, more preferably 90-99% or 94-99% of the second coagulant derived from or of Camelus dromedarius or Camelus bactrianus or Bos taurus, even more preferably 95-98% of the second coagulant derived from or of Camelus dromedarius or Camelus bactrianus or Bos taurus or 98-99% of the second coagulant derived from or of Camelus dromedarius or Camelus bactrianus or Bos taurus, wherein the % indicates the IMCU/ml of the second coagulant relative to the IMCU/ml of total coagulants in the composition. Preferably, the second coagulant derived from or of Camelus dromedarius or Camelus bactrianus may have at least 80% sequence identity with SEQ ID NO: 3 or 4 or 5, preferably at least 85% or at least 90% or at least 91% or at least 92% or at least 93% or at least 94% or at least 95% or at least 96% or at least 97% or at least 98% or at least 99% sequence identity with SEQ ID NO: 3 or 4 or 5. Preferably, the second coagulant derived from or of Bos taurus may have at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7.

### Second Aspect of the Invention

Another aspect of this invention relates to a formulation of the above-mentioned composition, wherein said formulation promotes the stability over time and favors co-existence by attenuating catalysis of both coagulants.

Different causes of activity loss can be envisioned. Formulation may favor stability of a first coagulant while simultaneously leading to instability and activity loss during storage of a second coagulant. However, blending of two coagulants with low specificity may also result in mutual proteolytic degradation of the two i.e. one of the coagulants (protease) may use the other coagulant as substrate. Therefore, it is also an objective of the present invention to identify conditions or formulations that stabilize the structure of both coagulants, and favor its co-existence by attenuating its catalysis in the formulation.

This aspect may be challenging as each coagulant is individually marketed in formulations which may be significantly different between them. For example, an endothiapepsin such as THERMOLASE® from Chr. Hansen A/S may be formulated in 50% w/w glycerol and pH 4.5, while a camel chymosin, such as CHY-MAX® Supreme from Chr. Hansen A/S, may be formulated in 12% w/v NaCl (2 M) and pH 5.7.

In a preferred embodiment, the composition now disclosed may comprise a polyol selected from glycerol, sorbitol, monopropylene glycerol, sucrose, glucose, lactose or galactose. Preferably, the composition now disclosed may comprise 35-75% w/w or 36-75% w/w of glycerol, more preferably 40-60% w/w of glycerol or 50-60% w/w of glycerol, wherein % w/w indicates the weight of glycerol relative to total weight of the composition.

In a preferred embodiment, the composition now disclosed may comprise 25-65% w/w or 25-64% w/w of the first coagulant and the second coagulant, preferably 40-60% w/w or 40-50% w/w, wherein % w/w indicates the weight of the first coagulant and the second coagulant relative to total weight of the composition.

In a preferred embodiment, the composition now disclosed may have a pH of 4.5-5.5, preferably 5-5.5 or 4.5-5.0.

In a preferred embodiment, the composition now disclosed may comprise 40-60% w/w of glycerol and a pH of 5-5.5, or 50-60% w/w of glycerol and a pH of 4.5-5.0, or 35-45% w/w of glycerol and a pH 4.5-5.0.

In a preferred embodiment of the second aspect of the invention, the first coagulant derived from or of Cryphonectria parasitica may have at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2.

In a preferred embodiment of the second aspect of the invention, the second coagulant derived from or of Camelus dromedarius or Camelus bactrianus may have at least 80% sequence identity with SEQ ID NO: 3 or 4 or 5, preferably at least 85% or at least 90% or at least 91% or at least 92% or at least 93% or at least 94% or at least 95% or at least 96% or at least 97% or at least 98% or at least 99% sequence identity with SEQ ID NO: 3 or 4 or 5.

In a preferred embodiment of the second aspect of the invention, the second coagulant derived from or of Bos taurus may have at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7.

In a preferred embodiment of the second aspect of the invention, the solution to stabilize the structure of both coagulants, and favor its co-existence by attenuating its catalysis in the formulation may be obtained with a composition, in particular any composition, as disclosed in the first aspect of the invention comprising a polyol selected from glycerol, sorbitol, monopropylene glycerol, sucrose, glucose, lactose or galactose, such as with 35-75% w/w or 36-75% w/w of glycerol, more preferably 40-60% w/w of glycerol or 50-60% w/w of glycerol, wherein % w/w indicates the weight of glycerol relative to total weight of the composition. Alternatively or additionally, a composition, in particular any composition, as disclosed in the first aspect of the invention may comprise with 25-65% w/w or 25-64% w/w of the first coagulant and the second coagulant, preferably 40-60% w/w or 40-50% w/w, wherein % w/w indicates the weight of the first coagulant and the second coagulant relative to total weight of the composition and 35-75% w/w or 36-75% w/w of glycerol, more preferably 40-60% w/w of glycerol or 50-60% w/w of glycerol, wherein % w/w indicates the weight of glycerol relative to total weight of the composition. Alternatively or additionally to the above mentioned in this paragraph, a composition, in particular any composition, as disclosed in the first aspect of the invention may have a pH of pH of 4.5-5.5, preferably 5-5.5 or 4.5-5.0. Alternatively or additionally to the above mentioned in this paragraph, a composition, in particular any composition, as disclosed in the first aspect of the invention may comprise 40-60% w/w of glycerol and a pH of 5-5.5, or 50-60% w/w of glycerol and a pH of 4.5-5.0, or 35-45% w/w of glycerol and a pH 4.5-5.0.

### Third and Fourth Aspects of the Invention

The third aspect of the invention relates to a method for making a milk-based product comprising adding an effective amount of the composition herein disclosed, in particular in the first and second aspects of the invention, and carrying out further manufacturing steps to obtain the milk-based product.

The fourth aspect of the invention relates to a method for making a milk-based product comprising the following steps:
adding a first coagulant derived from or of Cryphonectria, preferably Cryphonectria parasitica; and a second coagulant derived from or of Camelus, preferably Camelus dromedarius or Camelus bactrianus, more preferably Camelus dromedaries, or a second coagulant derived from or of Bos, preferably Bos taurus;
wherein the strength of the first coagulant added is 1-25 IMCU/Lₘᵢₗₖ and the strength of the second coagulant added is 32-51 IMCU/Lₘᵢₗₖ; and
carrying out manufacturing steps to obtain the milk-based product.

Alternatively, the fourth aspect of the invention relates to a method for making a milk-based product comprising the following steps:
adding a first coagulant and a second coagulant, wherein the first coagulant has at least 80% or least 85% or least 90% or at least 91% or at least 92% or at least 93% or at least 94% or at least 95% or at least 96% or at least 97% or at least 98% or at least 99% or 100% sequence identity with SEQ ID NO: 1 or 2 and the second coagulant has at least 80% or least 85% or least 90% or at least 91% or at least 92% or at least 93% or at least 94% or at least 95% or at least 96% or at least 97% or at least 98% or at least 99% or 100% sequence identity with SEQ ID NO: 3 or 4 or 5 or 6 or 7.

In a preferred embodiment of the fourth aspect of the invention, the strength of the first coagulant is 2-15 IMCU/Lₘᵢₗₖ and the strength of the second coagulant is 37-50 IMCU/Lₘᵢₗₖ, preferably wherein the strength of the first coagulant is 3-12 IMCU/Lₘᵢₗₖ and the strength of the second coagulant is 40-49 IMCU/Lₘᵢₗₖ.

In a preferred embodiment of the fourth aspect of the invention, the milk-based product may be cheese, preferably the milk-based product may be Cheddar cheese or Continental cheese or Swiss type cheese.

In a preferred embodiment of the fourth aspect of the invention, the first coagulant derived from or of Cryphonectria, preferably Cryphonectria parasitica, may be an endothiapepsin.

In a preferred embodiment of the fourth aspect of the invention, the first coagulant derived from or of Cryphonectria, preferably Cryphonectria parasitica, may have at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2, more preferably wherein the first coagulant is SEQ ID NO: 1 or 2 or a genetically-engineered coagulant derived from SEQ ID NO: 1 or 2. In another preferred embodiment, the genetically-engineered coagulant derived from SEQ ID NO: 1 or 2 has at least 80% sequence identity with SEQ ID NO: 1 or 2, preferably at least 85% or at least 90% or at least 91% or at least 92% or at least 93% or at least 94% or at least 95% or at least 96% or at least 97% or at least 98% or at least 99% sequence identity with SEQ ID NO: 1 or 2.

In a preferred embodiment of the fourth aspect of the invention, the first coagulant derived from or of Cryphonectria, preferably Cryphonectria parasitica, may be an encapsulated coagulant.

In a preferred embodiment of the fourth aspect of the invention, the first coagulant derived from or of Cryphonectria, preferably Cryphonectria parasitica, may be an endothiapepsin or an encapsulated endothiapepsin.

In a preferred embodiment of the fourth aspect of the invention, the second coagulant derived from or of Camelus may have at least 80% sequence identity with SEQ ID NO: 3 or 4 or 5, preferably at least 85% or at least 90% or at least 91% or at least 92% or at least 93% or at least 94% or at least 95% or at least 96% or at least 97% or at least 98% or at least 99% sequence identity with SEQ ID NO: 3 or 4 or 5, more preferably wherein the second coagulant is SEQ ID NO: 3 or 4 or 5, or a genetically-engineered coagulant derived from SEQ ID NO: 3 or 4 or 5. In a more preferred embodiment, the second coagulant derived from or of Camelus or a genetically-engineered coagulant derived from SEQ ID NO: 3 or 4 or 5 may be any of the sequences SEQ ID NO: 8-52. Any of these sequences may be equally chosen, in particular as these sequences correspond to high C/P ratio coagulants, in particular derived from camel.

In a preferred embodiment of the fourth aspect pf the invention, the second coagulant derived from or of Bos may have at least 80% sequence identity with SEQ ID NO: 6 or 7, preferably at least 85% or at least 90% or at least 91% or at least 92% or at least 93% or at least 94% or at least 95% or at least 96% or at least 97% or at least 98% or at least 99% sequence identity with SEQ ID NO: 6 or 7, more preferably wherein the second coagulant is SEQ ID NO: 6 or 7, or a genetically-engineered coagulant derived from SEQ ID NO: 6 or 7.

### Fifth Aspect of the Invention

Another aspect of the invention relates to the use of a composition as herein disclosed, in particular in the first and second aspects of the invention, for a process of making milk-based product, preferably for a process of making cheese, more preferably for a process of making Cheddar cheese or Continental cheese or Swiss type cheese. The composition is disclosed in detail in the first and second aspects above and in the examples below. All the features of the composition disclosed in the above aspects are also present in the fifth aspect of the invention.

### Sixth Aspect of the Invention

Another aspect of the invention relates to the use of a composition as herein disclosed, in particular in the first and second aspects of the invention, as an increaser of yield in cheese curd and/or as an increaser of cheese yield and/or as an increaser of proteolysis after cheese making and/or as an increaser of proteolysis during cheese ripening.

All the features of the composition disclosed in the above aspects are also present in the sixth aspect of the invention.

### Definitions

All definitions of herein relevant terms are in accordance of what would be understood by the skilled person in relation to the herein relevant technical context.

The term “coagulant blend” or “blend” relates to blends of enzymes used to coagulate milk, e.g. in a cheese making process. A blend of coagulating enzymes is a composition for clotting milk comprising at least two coagulant enzymes or at least two milk clotting enzymes.

The term “chymosin activity” relates to chymosin activity of a chymosin enzyme as understood by the skilled person in the present context. The skilled person knows how to determine herein relevant chymosin activity.

As known in the art—chymosin specificity may be determined by the so-called C/P ratio, which is determined by dividing the specific clotting activity (C) with the proteolytic activity (P). As known in the art—a higher C/P ratio implies generally that the loss of protein during e.g. cheese manufacturing due to non-specific protein degradation is reduced, i.e. the yield of cheese is improved. The detailed description provides an example of a standard method to determine specific chymosin activity—alternatively termed clotting activity or milk clotting activity. As an example the clotting activity may be determined using the REMCAT method, which is the standard method developed by the International Dairy Federation (IDF 157 or ISO 11815|IDF 157:2007).

The term “IMCU” means International Milk-Clotting Units. In the context of the present invention “IMCU/L of milk” corresponds to the dosage or strength recommended for cheese making, while “IMCU/ml” corresponds to the average activity of a commercial coagulant or commercial composition of coagulants.

The term “mature polypeptide” means a peptide in its final form following translation and any post-translational modifications, such as N-terminal processing, C-terminal truncation, glycosylation, phosphorylation, etc. In the present context, a mature chymosin polypeptide is the active chymosin polypeptide sequence—i.e. without the pre-part and/or pro-part sequences.

The term “Sequence Identity” relates to the relatedness between two amino acid sequences or between two nucleotide sequences and may calculated according to the methods available to the person skilled in the art. For purposes of the present invention, the degree of sequence identity between two amino acid sequences may be determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

The term “isolated variant” or “variant” or “chymosin variant” means a variant that is modified by the act of man and having improved chymosin activity when compared to the wild-type chymosin peptide from each it derives.

The term “wild-type” chymosin peptide means a chymosin expressed by a naturally occurring organism, such as e.g. a mammalian (e.g. camel or bovine) found in nature.

A liquid formulation of a coagulant when stored at 5° C. is considered stable when relative activity loss in 1 year is no more than 5%.

The term “first coagulant derived from Cryphonectria” or “first coagulant derived from Cryphonectria, preferably derived from Cryphonectria parasitica” means a first coagulant having at least 80%, at least 85%, at least 90%, at least 85% or at least 91% or at least 92% or at least 93% or at least 94% or at least 95% or at least 96% or at least 97% or at least 98% or at least 99% or 100% sequence identity with SEQ ID NO: 1 or 2, which may include a genetically-engineered coagulant of SEQ ID NO: 1 or 2.

The term “second coagulant derived from Camelus” or “second coagulant derived from Camelus, preferably derived from Camelus dromedarius or Camelus bactrianus” means a second coagulant having at least 80%, at least 85%, at least 90%, at least 85% or at least 91% or at least 92% or at least 93% or at least 94% or at least 95% or at least 96% or at least 97% or at least 98% or at least 99% or 100% sequence identity with SEQ ID NO: 3 or 4 or 5, which may include a genetically-engineered coagulant of SEQ ID NO: 3 or 4 or 5.

The term “second coagulant derived from Bos” or “second coagulant derived from Bos, preferably derived from Bos taurus” means a second coagulant having at least 80%, at least 85%, at least 90%, at least 85% or at least 91% or at least 92% or at least 93% or at least 94% or at least 95% or at least 96% or at least 97% or at least 98% or at least 99% or 100% sequence identity with SEQ ID NO: 6 or 7, which may include a genetically-engineered coagulant of SEQ ID NO: 6 or 7.

In the context of the present invention, a chymosin having a C/P ratio higher than the camel chymosin may be used instead of the camel chymosin. Therefore, CHY-MAX® M or SEQ ID NO: 3 or SEQ ID NO: 4 or SEQ ID NO: 5 may be replaced by a chymosin having a higher C/P ratio than CHY-MAX® M or SEQ ID NO: 3 or SEQ ID NO: 4 or SEQ ID NO: 5.

In the context of the present invention, a chymosin having a C/P ratio higher than the bovine chymosin may be used instead of the bovine chymosin. Therefore, CHY-MAX® or SEQ ID NO: 6 or SEQ ID NO: 7 may be replaced by a chymosin having a higher C/P ratio than CHY-MAX® or SEQ ID NO: 6 or SEQ ID NO: 7.

### BRIEF DESCRIPTION OF THE FIGURES

FIG. 1. Cheese yield expressed as gram dry matter (g DM) after subtraction of the moisture measured at 1-month (1M) and 4-months (4M) of ripening from the initial wet mass of the cheese obtained on the day of making cheese, obtained when compositions 1, 2 and 3 are used in the cheese making process.

FIG. 2. Fat content measured by MilkoScan™ from FOSS Analytics in the whey collected on the day of making the cheese, obtained when compositions 1, 2 and 3 are used in the cheese making process.

FIG. 3. Fat in dry matter (FDM) of the cheese blocks at 1-month (1M) and 4-months (4M) of ripening as measured by Foodscan™ from FOSS Analytics, obtained when compositions 1˜4 are used in the cheese making process.

FIG. 4. Firmness over time of cheese made using compositions 5-8.

FIG. 5. Alpha-casein over time of cheese made using compositions 5-8.

FIG. 6. Beta-casein over time of cheese made using compositions 5-8.

FIG. 7. Alpha-casein/Beta-casein ratio over time of cheese made using compositions 5-8.

FIG. 8. Firmness over time of compositions 9-16.

FIG. 9. Total casein over time for compositions 9-16.

FIG. 10. Alpha-casein over time for compositions 9-16.

FIG. 11. Beta-casein over time for compositions 9-16.

FIG. 12. Alpha-casein/Beta-casein over time for compositions 9-16.

FIG. 13. Total casein versus SN/TN at 30 days for composition 9-16.

FIG. 14A-I. Relative activity during storage at 5° C. of blends of THERMOLASE® and CHY-MAX® Supreme, both from Chr. Hansen, made with different formulations.

FIG. 15. A. Stability of blends versus calculated content of glycerol and B. Stability of blends versus content of NaCl.

FIG. 16A-D. Stability of blends of THERMOLASE® and CHY-MAX® Supreme. both from Chr. Hansen A/S, studied in factorial design with glycerol at levels 40, 50 and 60% and pH at levels 4.5, 5.0 and 5.5.

### BRIEF DESCRIPTION OF THE SEQUENCES

(endothiapepsin with propeptide)
SEQ ID NO: 1
MSSPLKNALVTAMLAGGALSSPTKQHVGIPVNASPEVGPGKYSFKQVRNPNYKFNGPLSVKKTYLKYGVPIPAWLEDAVQNST
SGLAERSTGSATTTPIDSLDDAYITPVQIGTPAQTLNLDFDTGSSDLWVFSSETTASEVDGQTIYTPSKSTTAKLLSGATWSI
SYGDGSSSSGDVYTDTVSVGGLTVTGQAVESAKKVSSSFTEDSTIDGLLGLAFSTLNTVSPTQQKTFFDNAKASLDSPVFTAD
LGYHAPGTYNFGFIDTTAYTGSITYTAVSTKQGFWEWTSTGYAVGSGTEKSTSIDGIADTGTTLLYLPATVVSAYWAQVSGAK
SSSSVGGYVFPCSATLPSFTFGVGSARIVIPGDYIDFGPISTGSSSCFGGIQSSAGIGINIFGDVALKAAFVVENGATTPTLG
FASK
(endothiapepsin w/o propeptide)
SEQ ID NO: 2
STGSATTTPIDSLDDAYITPVQIGTPAQTLNLDFDTGSSDLWVFSSETTASEVDGQTIYTPSKSTTAKLLSGATWSISYGDGS
SSSGDVYTDTVSVGGLTVTGQAVESAKKVSSSFTEDSTIDGLLGLAFSTLNTVSPTQQKTFFDNAKASLDSPVFTADLGYHAP
GTYNFGFIDTTAYTGSITYTAVSTKQGFWEWTSTGYAVGSGTEKSTSIDGIADTGTTLLYLPATVVSAYWAQVSGAKSSSSVG
GYVFPCSATLPSFTFGVGSARIVIPGDYIDFGPISTGSSSCFGGIQSSAGIGINIFGDVALKAAFVVENGATTPTLGFASK
(camel chymosin-Camelus dromedarius)
SEQ ID NO: 3
MRCLVVLLAALALSQASGITRIPLHKGKTLRKALKERGLLEDFLQRQQYAVSSKYSSLGKVAREPLTSYLDSQYFGKIYIGTP
PQEFTVVFDTGSSDLWVPSIYCKSNVCKNHHRFDPRKSSTFRNLGKPLSIHYGTGSMEGFLGYDTVTVSNIVDPNQTVGLSTE
QPGEVFTYSEFDGILGLAYPSLASEYSVPVFDNMMDRHLVARDLFSVYMDRNGQGSMLTLGAIDPSYYTGSLHWVPVTLQQYW
QFTVDSVTINGVAVACVGGCQAILDTGTSVLFGPSSDILKIQMAIGATENRYGEFDVNCGNLRSMPTVVFEINGRDYPLSPSA
YTSKDQGFCTSGFQGDNNSELWILGDVFIREYYSVFDRANNRVGLAKAI
(mature camel chymosin-Camelus dromedarius)
SEQ ID NO: 4
GKVAREPLTSYLDSQYFGKIYIGTPPQEFTVVFDTGSSDLWVPSIYCKSNVCKNHHRFDPRKSSTERNLGKPLSIHYGTGSME
GFLGYDTVTVSNIVDPNQTVGLSTEQPGEVFTYSEFDGILGLAYPSLASEYSVPVFDNMMDRHLVARDLFSVYMDRNGQGSML
TLGAIDPSYYTGSLHWVPVTLQQYWQFTVDSVTINGVAVACVGGCQAILDTGTSVLFGPSSDILKIQMAIGATENRYGEFDVN
CGNLRSMPTVVFEINGRDYPLSPSAYTSKDQGFCTSGFQGDNNSELWILGDVFIREYYSVFDRANNRVGLAKAI
(camel chymosin -Camelus dromedarius)
SEQ ID NO: 5
MRCLVVLLAALALSQASGITRIPLHKGKTLRKALKERGLLEDFLQRQQYAVSSKYSSLGKVAREPLTSYLDSQYFGKIYIGTP
PQEFTVVFDTGSSDLWVPSIYCKSNACKNHHRFDPRKSSTFRNLGKPLSIHYGTGSIEGFLGYDTVTVSNIVDPNQTVGLSTE
QPGEVFTYSEFDGILGLAYPSLASEYSVPVFDNMMDRHLVARDLFSVYMDRNGQGSMLTLGATDPSYYTGSLHWVPVTVQQYW
QVTVDSVTINGVAVACVGGCQAILDTGTSVLFGPSSDILKIQMAIGATENRYGEFDVNCGSLRSMPTVVFEINGRDFPLAPSA
YTSKDQGFCTSGFQGDNNSELWILGDVFIREYYSVFDRANNRVGLAKAI
(bovine chymosin-Bos taurus)
SEQ ID NO: 6
MRCLVVLLAVFALSQGAEITRIPLYKGKSLRKALKEHGLLEDFLQKQQYGISSKYSGFGEVASVPLTNYLDSQYFGKIYLGTP
PQEFTVLFDTGSSDFWVPSIYCKSNACKNHQRFDPRKSSTFQNLGKPLSIHYGTGSMQGILGYDTVTVSNIVDIQQTVGLSTQ
EPGDVFTYAEFDGILGMAYPSLASEYSIPVFDNMMNRHLVAQDLFSVYMDRNGQESMLTLGAIDPSYYTGSLHWVPVTVQQYW
QFTVDSVTISGVVVACEGGCQAILDTGTSKLVGPSSDILNIQQAIGATQNQYGEFDIDCDNLSYMPTVVFEINGKMYPLTPSA
YTSQDQGFCTSGFQSENHSQKWILGDVFIREYYSVEDRANNLVGLAKAI
(mature bovine chymosin -Bos taurus)
SEQ ID NO: 7
GEVASVPLTNYLDSQYFGKIYLGTPPQEFTVLFDTGSSDFWVPSIYCKSNACKNHQRFDPRKSSTFQNLGKPLSIHYGTGSMQ
GILGYDTVTVSNIVDIQQTVGLSTQEPGDVFTYAEFDGILGMAYPSLASEYSIPVEDNMMNRHLVAQDLFSVYMDRNGQESML
TLGAIDPSYYTGSLHWVPVTVQQYWQFTVDSVTISGVVVACEGGCQAILDTGTSKLVGPSSDILNIQQAIGATQNQYGEFDID
CDNLSYMPTVVFEINGKMYPLTPSAYTSQDQGFCTSGFQSENHSQKWILGDVFIREYYSVEDRANNLVGLAKAI

Alternatively, SEQ ID NO: 1 and SEQ ID NO: 2 may correspond to THERMOLASE® from Chr. Hansen A/S or Suparen® from DSM. Alternatively, SEQ ID NO: 1 and SEQ ID NO: 2 may have an optimized sequence such as an optimized N-terminal sequence.

Alternatively, SEQ ID NO: 3 may be replaced by any chymosin (or chymosin variant) having improved chymosin activity, as disclosed in WO2016207214, WO2017198810 or WO2017198829 showing improved chymosin activity or improved C/P ratio or by SEQ ID NO: 5. Further, any chymosin disclosed in WO2016207214, WO2017198810 or WO2017198829 is herein included by reference provided its shows improved chymosin activity or improved C/P ratio, for example, variants 417-461 (numbering used in WO2016207214, WO2017198810 or WO2017198829) herein represented by SEQ ID NO: 8-52. Further, in the present patent document SEQ ID NO: 3, SEQ ID NO: 4, SEQ ID NO: 5, camel chymosin, CHY-MAX® M and CHY-MAX® Supreme may be interchangeably used.

Alternatively, SEQ ID NO: 6 or 7 may be replaced by any sequence having improved chymosin activity, for example, any sequence disclosed in WO2013164479 or WO2013164481, showing improved chymosin activity. Further, in the present patent document, SEQ ID NO: 6, SEQ ID NO: 7 and bovine chymosin may be interchangeably used.

>SEQ ID NO: 8
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNIVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVIFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 9
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVIFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 10
GKVAREPLTSILDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVIFGPSSDILKIQMAIGATENEYGEFDVE
CDNLRSMPTVVFEINGRDYPLSPSAYTSKDQGFCTSGFQGDNNSELWILGDVFIREYYSVEDRANNRVGLAKAI
>SEQ ID NO: 11
GKVAREPLTSILDSQYFGTIYIGTPPQEFTVVFDTGSSDLWVPSIYCKSNVCKNHHRENPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVIFGPSSDILKIQMAIGATENEYGEFDVN
CDNLRSMPTVVFEINGRDYPLSPSAYTSKDQGFCTSGFQGDNNSELWILGDVFIREYYSVEDRANNRVGLAKAI
>SEQ ID NO: 12
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGSMV
TLGAIDPSYYTGSLHWVPVTLQQYWQFTVDSVTINGVAVACVGGCQAILDTGTSVVFGPSSDILKIQMAIGATENEYGEFDVE
CDNIRSMPTVVFEINGRDYPLSPSAYTSKDQGFCTSGFQGDNNSELWILGDVFIREYYSVEDRANNRVGLAKAI
>SEQ ID NO: 13
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVLFGPSSDILKIQMAIGATENEYGEFDVN
CGNLRSMPTVVFEINGRDYPLSPSAYTSKDQGFCTSGFQGDNNSELWILGDVFIREYYSVFDRANNRVGLAKAI
>SEQ ID NO: 14
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVEDRANNRVGLAKAI
>SEQ ID NO: 15
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVEDNMMDRHLVARDLFSVYMDRNGQGGMI
TLGAIDPSYYTGSLHWVPVTLQQYWQFTVDSVTINGVAVACVGGCQAILDTGTSVLFGPSSDILKIQMAIGATENEYGEFDVE
CDNIRSMPTVVFEINGRDYPLSPSAYTSKDQGFCTSGFQGDNNSELWILGDVFIREYYSVFDRANNRVGLAKAI
>SEQ ID NO: 16
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVEDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CGNLRSMPTVVFEINGRDYPLSPSAYTSKDQGFCTSGFQGDNNSELWILGDVFIREYYSVFDRANNRVGLAKAI
>SEQ ID NO: 17
GKVAREPLTSVLDSQYFGTIYIGTPPQEFTVVEDTGSSDLWVPSIYCKSNVCKNHHRFNPRKSSTERNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVE
CDNIRSMPTVVFEINGRDYPLSPSAYTSKDQGFCTSGFQGDNNSELWILGDVFIREYYSVEDRANNRVGLAKAI
>SEQ ID NO: 18
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNIVDPNQTVGLSTEQPGEVFTYSEFDGILGLAYPSLASEYSVPVFDNMMDRHLVARDLFSVYMDRNGQGSMV
TLGAIDPSYYTGSLHWVPVTLQQYWQFTVDSVTINGVAVACVGGCQAILDTGTSVIFGPSSDILKIQMAIGATENEYGEFDVE
CDNIRSMPTVVFEINGRDYPLSPSAYTSKDQGFCTSGFQGDNNSELWILGDVFIREYYSVEDRANNRVGLAKAI
>SEQ ID NO: 19
GKVAREPLTSVLDSQYFGTIYIGTPPQEFTVVFDTGSSDLWVPSIYCKSNVCKNHHRFNPRKSSTERNLGKPLSIHYGTGSME
GFLGYDTVTVSNLVDPNQTVGLSTEQPGEVFTYSEFDGILGLAYPSLASEYSVPVFDNMMDRHLVARDLESVYMDRNGQGGMV
TLGAIDPSYYTGSLHWVPVTLQQYWQFTVDSVTINGVAVACVGGCQAILDTGTSVIFGPSSDILKIQMAIGATENEYGEFDVE
CGNLRSMPTVVFEINGRDYPLSPSAYTSKDQGFCTSGFQGDNNSELWILGDVFIREYYSVEDRANNRVGLAKAI
>SEQ ID NO: 20
GKVAREPLTSVLDSQYFGTIYIGTPPQEFTVVFDTGSSDLWVPSIYCKSNVCKNHHRFNPRKSSTERNLGKPLSIHYGTGSME
GFLGYDTVTVSNIVDPNQTVGLSTEQPGEVFTYSEFDGILGLAYPSLASEYSVPVFDNMMDRHLVARDLESVYMDRNGQGGMI
TLGAIDPSYYTGSLHWVPVTLQQYWQFTVDSVTINGVAVACVGGCQAILDTGTSVIFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 21
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVEDNMMDRHLVARDLESVYMDRNGQGGML
TLGAIDPSYYTGSLHWVPVTLQQYWQFTVDSVTINGVAVACVGGCQAILDTGTSVLFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 22
GKVAREPLTSVLDSQYFGKIYIGTPPQEFTVVFDTGSSDLWVPSIYCKSNVCKNHHRFNPRKSSTERNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVN
CDNIRSMPTVVFEINGRDYPLSPSAYTSKDQGFCTSGFQGDNNSELWILGDVFIREYYSVEDRANNRVGLAKAI
>SEQ ID NO: 23
GKVAREPLTSVLDSQYFGKIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVIFGPSSDILKIQMAIGATENEYGEFDVN
CDNLRSMPTVVFEINGRDYPLSPSAYTSKDQGFCTSGFQGDNNSELWILGDVFIREYYSVEDRANNRVGLAKAI
>SEQ ID NO: 24
GKVAREPLTSILDSQYFGKIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVEDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVN
CDNIRSMPTVVFEINGRDYPLSPSAYTSKDQGFCTSGFQGDNNSELWILGDVFIREYYSVFDRANNRVGLAKAI
>SEQ ID NO: 25
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVIFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 26
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVEDRANNRVGLAKAI
>SEQ ID NO: 27
GKVAREPLTSVLDSQYFGTIYIGTPPQEFTVVFDTGSSDLWVPSIYCKSNVCKNHHRENPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CGNIRSMPTVVFEINGRDYPLSPSAYTSKDQGFCTSGFQGDNNSELWILGDVFIREYYSVEDRANNRVGLAKAI
>SEQ ID NO: 28
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 29
GKVAREPLTSILDSQYFGTIYIGTPPQEFTVVFDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVLFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 30
GKVAREPLTSVLDSQYFGTIYIGTPPQEFTVVFDTGSSDLWVPSIYCKSNVCKNHHRENPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 31
GKVAREPLTSVLDSQYFGTIYIGTPPQEFTVVEDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 32
GKVAREPLTSILDSQYFGTIYIGTPPQEFTVVFDTGSSDLWVPSIYCKSNVCKNHHRFDPRKSSTFRNLGKPLSIHYGTGSME
GFLGYDTVTVSNIVDPNQTVGLSTEQPGEVFTYSEFDGILGLAYPSLASEYSVPVFDNMMDRHLVARDLFSVYMDRNGQGSML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVEDRANNRVGLAKAI
>SEQ ID NO: 33
GKVAREPLTSVLDSQYFGTIYIGTPPQEFTVVEDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVEDNMMDRHLVARDLFSVYMDRNGQGSML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVEDRANNRVGLAKAI
>SEQ ID NO: 34
GKVAREPLTSILDSQYFGTIYIGTPPQEFTVVEDTGSSDLWVPSIYCKSNVCKNHHRENPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 35
GKVAREPLTSVLDSQYFGTIYIGTPPQEFTVVFDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 36
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 37
GKVAREPLTSILDSQYFGKIYIGTPPQEFTVVFDTGSSDLWVPSIYCKSNVCKNHHRFDPRKSSTERNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVEDRANNRVGLAKAI
>SEQ ID NO: 38
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVVFGPSSDILKIQMAIGATENEYGEFDVE
CGNLRSMPTVVFEINGRDYPLSPSAYTSKDQGFCTSGFQGDNNSELWILGDVFIREYYSVFDRANNRVGLAKAI
>SEQ ID NO: 39
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGSML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 40
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVIFGPSSDILKIQMAIGATENEYGEFDVN
CGNLRSMPTVVFEINGRDYPLSPSAYTSKDQGFCTSGFQGDNNSELWILGDVFIREYYSVFDRANNRVGLAKAI
>SEQ ID NO: 41
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLESVYMDRNGQGGMV
TLGAIDPSYYTGSLHWVPVTLQQYWQFTVDSVTINGVAVACVGGCQAILDTGTSVLFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 42
GKVAREPLTSILDSQYFGTIYIGTPPQEFTVVFDTGSSDLWVPSIYCKSNVCKNHHRFNPRKSSTFRNLGKPLSIHYGTGSME
GFLGYDTVTVSNIVDPNQTVGLSTEQPGEVFTYSEFDGILGLAYPSLASEYSVPVFDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 43
GKVAREPLTSILDSQYFGKIYIGTPPQEFTVVFDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGSML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVEDRANNRVGLAKAI
>SEQ ID NO: 44
GKVAREPLTSILDSQYFGKIYIGTPPQEFTVVFDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVIFGPSSDILKIQMAIGATENEYGEFDVN
CGNLRSMPTVVFEINGRDYPLSPSAYTSKDQGFCTSGFQGDNNSELWILGDVFIREYYSVFDRANNRVGLAKAI
>SEQ ID NO: 45
GKVAREPLTSVLDSQYFGTIYIGTPPQEFTVVEDTGSSDLWVPSIYCKSNVCKNHHRFDPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGSMV
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVEDRANNRVGLAKAI
>SEQ ID NO: 46
GKVAREPLTSILDSQYFGKIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVIFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 47
GKVAREPLTSILDSQYFGKIYIGTPPQEFTVVEDTGSSDLWVPSIYCKSNVCKNHHRFNPRKSSTERNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 48
GKVAREPLTSILDSQYFGTIYIGTPPQEFTVVFDTGSSDLWVPSIYCKSNVCKNHHRENPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGML
TLGAIDPSYYTGSLHWVPVTLQQYWQFTVDSVTINGVAVACVGGCQAILDTGTSVIFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 49
GKVAREPLTSILDSQYFGKIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 50
GKVAREPLTSVLDSQYFGTIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGSML
TLGAIDPSYYTGSLHWVPVTLQQYWQFTVDSVTINGVAVACVGGCQAILDTGTSVVFGPSSDILKIQMAIGATENEYGEFDVN
CDNLRSMPTVVFEINGRDYPLSPSAYTSKDQGFCTSGFQGDNNSELWILGDVFIREYYSVFDRANNRVGLAKAI
>SEQ ID NO: 51
GKVAREPLTSILDSQYFGTIYIGTPPQEFTVVFDTGSSDLWVPSIYCKSNVCKNHHRFDPRKSSTERNLGKPLSIHYGTGSME
GFLGYDTVTVSNIVDPNQTVGLSTEQPGEVFTYSEFDGILGLAYPSLASEYSVPVEDNMMDRHLVARDLFSVYMDRNGQGGMI
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI
>SEQ ID NO: 52
GKVAREPLTSILDSQYFGKIYIGTPPQEFTVVFDTGSSDLWVPSIYCKSNVCKNHHRFNPRKSSTFRNLGKPLSIHYGTGSME
GFLGYDTVTVSNLVDPNQTVGLSTEQPGEVFTYSEFDGILGLAYPSLASEYSVPVFDNMMDRHLVARDLFSVYMDRNGQGGMV
TLGAIDPSYYTGSLHWVPVTLQQYWQFTVDSVTINGVAVACVGGCQAILDTGTSVVFGPSSDILKIQMAIGATENEYGEFDVE
CDNLRSMPTVVFEINGRDYPLSPSAYTSKDQGFCTSGFQGDNNSELWILGDVFIREYYSVFDRANNRVGLAKAI

### DETAILED DESCRIPTION OF THE INVENTION

### Determination of Milk Clotting Activity

Milk clotting activity may be determined using the REMCAT method, which is the standard method developed by the International Dairy Federation (IDF 157 or ISO 11815| IDF 157:2007).

Milk clotting activity is determined from the time needed for a visible flocculation of a standard milk substrate prepared from a low-heat, low fat milk powder with a calcium chloride solution of 0.5 g per liter (PH≈6.5). The clotting time of a rennet sample is compared to that of a reference standard having known milk-clotting activity and having the same enzyme composition by IDF Standard 110B as the sample. Samples and reference standards are measured under identical chemical and physical conditions. Variant samples are adjusted to approximately 3 IMCU/ml using an 84 mM acetic acid buffer pH 5.5. Hereafter, 200 μl enzyme preparation is added to 10 ml preheated milk (32° C.) in a glass test tube placed in a water bath, capable of maintaining a constant temperature of 32° C.+1° C. under constant stirring.

The total milk-clotting activity (strength) of a rennet is calculated in International Milk-Clotting Units (IMCU) per ml relative to a standard having the same enzyme composition as the sample according to the formula:

$$Strength in IMCU / ml = \frac{Sstandard \times Tstandard \times Dsample}{Dstandard \times Tsample}$$

Sstandard: The milk-clotting activity of the international reference standard for rennet.
Tstandard: Clotting time in seconds obtained for the standard dilution.
Dsample: Dilution factor for the sample
Dstandard: Dilution factor for the standard
Tsample: Clotting time in seconds obtained for the diluted rennet sample from addition of enzyme to time of flocculation.

### Determination of Total Protein Content

Total protein content may preferably be determined using the Pierce BCA Protein Assay Kit from Thermo Scientific following the instructions of the providers.

### Calculation of Specific Clotting Activity

Specific clotting activity (IMCU/mg total protein) was determined by dividing the clotting activity (IMCU/ml) by the total protein content (mg total protein per ml).

### Determination of Proteolytic Activity

General proteolytic activity was measured using azo casein as the substrate. One unit of protease activity is defined as the amount of enzyme that provides absorbance at 425 nm of 1.00 per minute at 30° C. under defined conditions. For the assay, equal volumes of gel filtered sample of 135 IMCU/ml and pH 6.5, and 5% azo casein at pH 6.5 was incubated in a 30° C. water bath for exactly 30 minutes after which the reaction was stopped by adding 1.5 ml 5% TCA while mixing. The reaction tube was cooled in ice bath and centrifuged until a clear supernatant was obtained. One ml of the supernatant was mixed with 2 ml 4 NaOH and the extinction measured spectrophotometrically at 425 nm.

### Determination of the C/P Ratio

The C/P ratio is calculated by dividing the clotting activity (C) with the proteolytic activity (P).

### Determination of Casein Degradation

There are several ways described in the prior art to determine casein degradation. For example as disclosed in Kim et al 2004. Another way to determine casein degration may be using the LabChip® electrophoresis system, in particular the LabChip® HT Protein Express Assay (PN 760499) in combination with the LabChip® GXII Touch™ Protein Characterization System, both from PerkinElmer Inc. Briefly, a set of standards of isolated caseins at a known concentration was used to establish a calibration curve. Subsequently, corresponding caseins were identified and quantified in cheese extract obtained from a cheese sample prepared according to the Examples. The output obtained is a concentration of alpha-casein and beta-casein, both in mg/g of cheese. The summed concentrations of the casein types (alpha-casein and beta-casein) is referred to as total casein. In cheese, the total casein decreases over time due to degradation and a result the soluble nitrogen (SN)/total nitrogen (TN) (%) increases. As a consequence, the primary proteolysis also increases. Thus, casein quantification allows to evaluate the primary cheese proteolysis. If required, the degree of casein degradation can be expressed as a fraction of total protein (CNdegradation (%)). Generally, the degree of casein degradation acts in a similar way as the SN/TN (%), meaning that it increases as the proteolysis proceeds over time.

### EXAMPLES

In the examples below, a composition (or stock composition) comprising at most 25% of a first coagulant derived from or of Cryphonectria and at least 75% of a second coagulant derived from derived from or of Camelus or Bos; wherein at most 25% of the first coagulant indicates the IMCU/ml of the first coagulant relative to the IMCU/ml of total coagulants in the composition and at least 75% of the second coagulant indicates the IMCU/ml of the second coagulant relative to the IMCU/ml of total coagulants. By using said composition (stock composition), the compositions below (compositions 1-16) with the indicated strengths were obtained.

In particular and for the sake of completeness, a dosage of coagulants (or strength) of 52 IMCU/Lₘᵢₗₖ in a vat of milk may correspond to:
a dosage of 3 IMCU/Lₘᵢₗₖ of the first coagulant and 49 IMCU/Lₘᵢₗₖ of the second coagulant, which corresponds to about 5-6% of the first coagulant (e.g. endothiapepsin) and 94-95% of the second coagulant (e.g. camel chymosin);
a dosage of 6 IMCU/Lₘᵢₗₖ of the first coagulant and 46 IMCU/Lₘᵢₗₖ of the second coagulant, which corresponds to a 12% of the first coagulant (e.g. endothiapepsin) and 88% of the second coagulant (e.g. camel chymosin);
a dosage of 12 IMCU/Lₘᵢₗₖ of the first coagulant and 40 IMCU/Lₘᵢₗₖ of the second coagulant, which corresponds to a 23% of the first coagulant (e.g. endothiapepsin) and 77% of the second coagulant (e.g. camel chymosin).

In particular and for the sake of completeness, a dosage of coagulants (or strength) of 40 IMCU/Lₘᵢₗₖ in a vat of milk may correspond to:
a dosage of 6 IMCU/Lₘᵢₗₖ of the first coagulant and 34 IMCU/Lₘᵢₗₖ of the second coagulant, which corresponds to a 15% of the first coagulant (e.g. endothiapepsin) and 85% of the second coagulant (e.g. camel chymosin).

### Example 1

Cheese, in particular Continental cheese, was produced in small cheese vat (10 L). The milk used for making cheese had 3.61% fat, 3.53% protein, 4.88% lactose and 9.04% Solids Not Fat (SNF). The cheese-making process is standard in the art.

The composition for clotting milk used in Example 1 comprised:
a camel chymosin (CHY-MAX® M)—composition 1;
a camel chymosin (CHY-MAX® M) and a non-encapsulated endothiapepsin (THERMOLASE®)—composition 2;
a camel chymosin (CHY-MAX® M) and an encapsulated endothiapepsin (THERMOLASER)—compositions 3 and 4.

CHY-MAX® M and THERMOLASE® are both from Chr. Hansen A/S.

TABLE 1
Strength in IMCU/L of milk of each coagulant used in each
composition. For each composition, two repeats were made.

| | | Non-encapsulated | Encapsulated |
|---|---|---|---|
| Composition for | Camel chymosin | endothiapepsin | endothiapepsin |
| clotting milk | (CHY-MAX ® M) | (THERMOLASE ®) | (THERMOLASE ®) |
| Composition 1 | 40 | 0 | 0 |
| Composition 2 | 40 | 12.5 | 0 |
| Composition 3 | 40 | 0 | 12.5 |
| Composition 4 | 40 | 0 | 5 |

First, the camel chymosin was added at an activity of 40 IMCU/L of milk and the incubation continued for 3 minutes with stirring for the non-encapsulated endothiapepsin or for 10 minutes for the encapsulated endothiapepsin. Next, non-encapsulated endothiapepsin (composition 2) or encapsulated endothiapepsin (composition 3 or composition 4) was added. Non-encapsulated endothiapepsin was added at an activity of 12.5 IMCU/L of milk and the incubation continued with stirring, followed by standard protocol for making continental cheese. Encapsulated endothiapepsin was added at an activity of 12.5 IMCU/L of milk (for standard dosage) and 5 IMCU/L of milk (for the lower dosage) and the incubation continued with stirring, followed by standard protocol for making continental cheese.

Coagulant blends were made 30 min before used. Alternatively, the coagulant blend can be made well in advance and be stored for at least 1 year prior to use.

Coagulants, in particular endothiapepsin, were encapsulated using the procedure described in WO2020229670.

The exact amount of enzyme stock solutions added in the respective vats (2 vats per composition) was as given in Table 2.

TABLE 2
Amounts of enzyme stock solutions used in Example 1.

| Composition | | | |
|---|---|---|---|
| for clotting | | Non-encapsulated | Encapsulated |
| milk/Cheese | Camel chymosin | endothiapepsin | endothiapepsin |
| ID | (CHY-MAX ® M) | (THERMOLASE ®) | (THERMOLASE ®) |
| Composition 1/ | 0.25 g/10 kg | — | — |
| Vats C-1; C-2 | milk | | |
| Composition 2 | 0.25 g/10 kg | 0.2 g/10 kg milk | — |
| Vats F-1; F-2 | milk | | |
| Composition 3 | 0.25 g/10 kg | — | 2.5 g/1 kg milk |
| Vats E-1; E-2 | milk | | |
| Composition 4 | 0.25 g/10 kg | — | 1 g/1 kg milk |
| Vats E-3; E-4 | milk | | |

The culture used for acidification was an appropriated culture, such as Flora™ C950 culture from Chr. Hansen A/S. Alternatively, other appropriate cultures may be used. The skilled person is aware of said alternatives. However, there are many more examples well known to the skilled person as alternative cultures that can be used in cheese making. There was no significant difference in the acidification of the milk in various vats (C-1, C-2, F-1, F-2, and E-1, E-2, E-3; E-4)—data not shown.

### Composition of Whey and Fat in Dry Matter (FDM)

Whey was collected on the day of the cheese making. The composition of the whey was determined on the same day as collected using MilkoScan™ from FOSS Analytics, which gave the quantity (%) of fat, protein, lactose and SNF in the whey. Other methods to determine the composition of the whey can alternatively be used. These methods are well known to the skilled person.

The cheese was ripened using a standard protocol. After 1-month (1M) and 4-months (4M) of ripening, the cheese blocks were analyzed using a Foodscan™ Dairy Analyser from FOSS Analytics to measure the content of moisture, protein, fat, FDM, moisture nonfat substance (MNFS), salt, salt in moisture phase (SM) and total solid (TS). Other methods to measure the content of moisture, protein, fat, FDM, MNFS, salt, SM and TS can alternatively be used. The methods to measure the content of moisture, protein, fat, FDM, MNFS, salt, SM and TS are well known to the skilled person.

Cheese Yield Estimation from DM Recovery

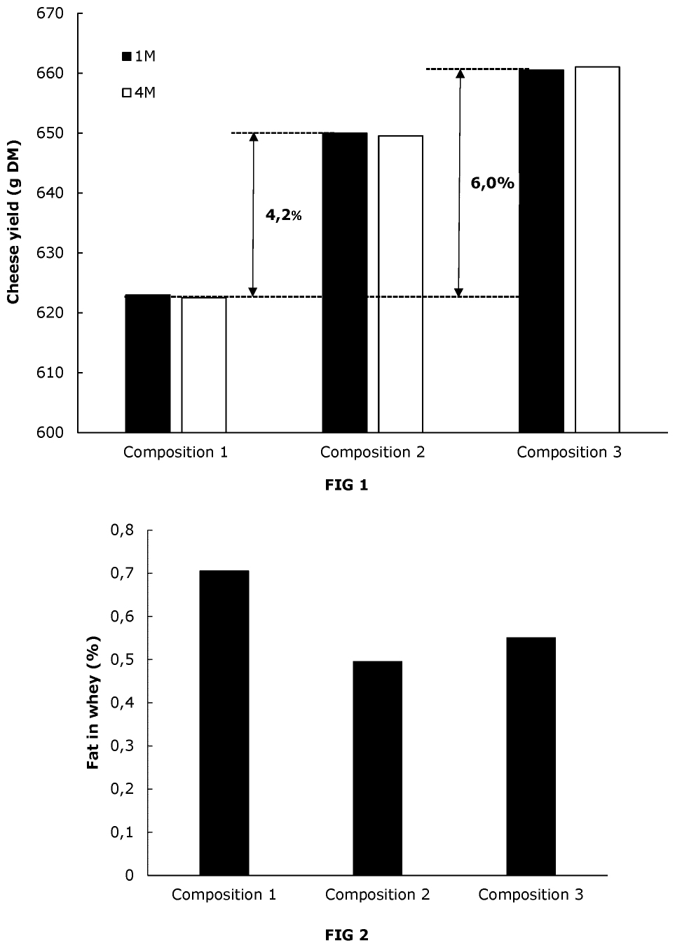

The cheese yield was calculated after correction for the moisture measured after 1M and 4M of ripening from the initial wet mass of the cheese obtained on the day of making cheese. The average and standard deviation was calculated from the duplicates (C-1, C-2 and F-1, F-2) and plotted to compare cheese yield (g DM)—FIG. 1.

FIG. 1 shows:
an increase in cheese yield of about 4.2% (g DM) is obtained when combining a camel chymosin and a non-encapsulated endothiapepsin (composition 2) versus the cheese yield obtained when only the camel chymosin (composition 1) was used for clotting the milk;
an increase in cheese yield of about 6.0% (g DM) is obtained when combining a camel chymosin and an encapsulated endothiapepsin (composition 3) versus the cheese yield obtained when only the camel chymosin (composition 1) was used for clotting the milk;
an increase in cheese yield of about 1.8% (g DM) is obtained when combining a camel chymosin and an encapsulated endothiapepsin (composition 3) versus the cheese yield obtained when combining a camel chymosin and a non-encapsulated endothiapepsin (composition 2).

In a preferred embodiment of the invention, an improvement in cheese yield can be obtained when a camel chymosin is combined with an endothiapepsin (compositions 2 or 3), regardless if the endothiapepsin is a non-encapsulated endothiapepsin (composition 2) or an encapsulated endothiapepsin (composition 3).

In a more preferred embodiment of the invention, an improvement in cheese yield can be obtained when a camel chymosin is combined with an encapsulated endothiapepsin (composition 3).

The increase in cheese yield leads to an increase in the amount of cheese produced by the cheese-making process.

### Fat in Whey and Fat in Dry Matter (FDM)

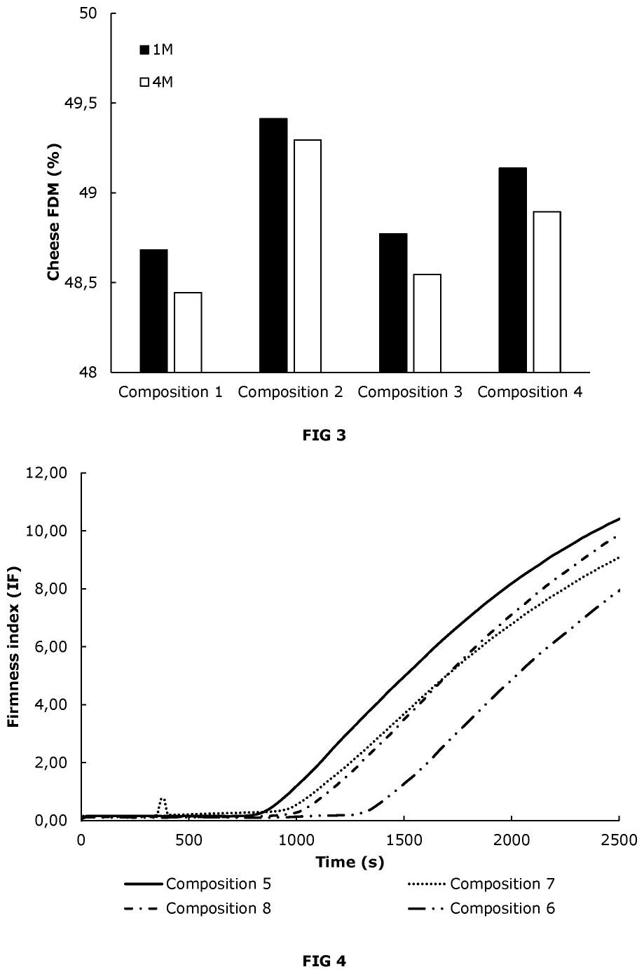

There was a significant difference in the fat content of the whey samples collected and measured on the same day of making the cheese when using compositions 2 and 3 (FIG. 2). These observations were found to correlate very well with the differences in the fat in dry matter (FDM) of the cheese blocks measured after 1-month (1M) and 4-months (4M) of ripening using Foodscan™ from FOSS Analytics (FIG. 3).

FIG. 3 shows:
an increase in FDM of about 1.5% or 1.8% is obtained for cheese ripened for 1-month (1M) or 4-months (4M), respectively, when combining a camel chymosin and a non-encapsulated endothiapepsin (composition 2) versus the FDM obtained when only the camel chymosin (composition 1) was used for clotting the milk;
an increase in FDM of about 0.2% was obtained when combining a camel chymosin and an encapsulated endothiapepsin (composition 3) versus the FDM obtained when only the camel chymosin (composition 1) was used for clotting the milk, regardless if the cheese is obtained after 1-month (1M) or 4-months (4M);
an increase in FDM of about 0.9% was obtained when combining a camel chymosin and an encapsulated endothiapepsin (composition 4) versus the FDM obtained when only the camel chymosin (composition 1) was used for clotting the milk, regardless if the cheese is obtained after 1-month (1M) or 4-months (4M);
an increase in FDM of about 1.3% or 1.5% is obtained for cheese ripened for 1-month (1M) or 4-months (4M), respectively, when combining a camel chymosin and a non-encapsulated endothiapepsin (composition 2) versus the FDM obtained when combining a camel chymosin and an encapsulated endothiapepsin (composition 3).
an increase in FDM of about 0.7% was obtained when combining a camel chymosin and a low dosage of an encapsulated endothiapepsin (composition 4) versus the FDM obtained when a camel chymosin and a higher dosage of an encapsulated endothiapepsin (composition 3) was used for clotting the milk, regardless if the cheese is obtained after 1-month (1M) or 4-months (4M).

In a preferred embodiment of the invention, an improvement in FDM can be obtained when a camel chymosin is combined with an endothiapepsin (compositions 2 or 3), regardless if the endothiapepsin is a non-encapsulated endothiapepsin (composition 2) or an encapsulated endothiapepsin (composition 3).

In a more preferred embodiment of the invention, an improvement in FDM can be obtained when a camel chymosin is combined with a non-encapsulated endothiapepsin (composition 2).

The increased FDM allows to reduce the fat content of the milk leading to cream saving. Thus, it creates more value from the raw material.

### Ripening

The proteolysis levels of cheeses produced by using compositions 1, 2 and 3 was also determined and are given in Table 3.

TABLE 3
Proteolysis (ST/TN) (%) at 1-month and at 4-months.

| | | At 1-month | | At 4-months | |
|---|---|---|---|---|---|
| Composition | | SN/ | Average | SN/ | Average |
| for clotting | | TN | SN/TN | TN | SN/TN |
| milk | Cheese ID | (%) | (%) | (%) | (%) |
| Composition 1 | C-1 | 9.71 | 9.71 | 20.58 | 20.69 |
| Composition 1 | C-2 | 9.70 | | 20.80 | |
| Composition 2 | F-1 | 15.99 | 16.08 | 27.93 | 27.81 |
| Composition 2 | F-2 | 16.18 | | 27.70 | |
| Composition 3 | E-1 | 16.24 | 16.28 | 25.83 | 25.96 |
| Composition 3 | E-2 | 16.31 | | 26.09 | |
| Composition 4 | E-3 | 14.97 | 15.31 | 27.09 | 27.71 |
| Composition 4 | E-4 | 15.65 | | 28.33 | |

Table 3 shows that, at 1-month (1M) and 4-months (4M) of ripening, the proteolysis (SN/TN) is higher when a combination of a camel chymosin and a non-encapsulated or an encapsulated endothiapepsin is used (compositions 2, 3 or 4), regardless of the dosage of non-encapsulated or encapsulated endothiapepsin, versus when only a camel chymosin is used (composition 1). Thus, for the same ripening time (1-month or 4-months), a combination of a camel chymosin and a non-encapsulated or an encapsulated endothiapepsin (compositions 2, 3 or 4) leads to improved proteolysis levels. Further, by using a combination of a camel chymosin and a non-encapsulated or an encapsulated endothiapepsin (compositions 2, 3 or 4) it is possible to reduce the ripening time. Finally, an acceptable value of proteolysis at 1-month and 4-months ranges from 13-17% and 20-30%, respectively. For compositions 2-4, all proteolysis values are within the acceptable range, both for 1-month and 4-months of ripening. In contrast, the proteolysis level when composition 1 is used for cheese making, is unacceptable at 1-month of ripening and close to the lower limit of 20% at 4-months of ripening.

In conclusion, Table 3 shows that the proteolysis levels are significantly improved when a composition for clotting milk comprises a camel chymosin blended with a endothiapepsin, either non-encapsulated endothiapepsin or encapsulated endothiapepsin.

Identical results are expected for other types of cheese, such as Cheddar cheese, any type of continental cheese, and Swiss cheese type. Further, identical results are also expected if THERMOLASE® from Chr. Hansen A/S is replaced by Suparen® from DSM or a similar endothiapepsin. Finally, identical results are also expected if the camel chymosin is replaced by a bovine chymosin.

### Example 2

Continental cheeses were produced in a small cheese vat (10 L each) with milk having a fat/protein ratio of 1. To produce said Continental cheese, the DVS® culture C960 from Chr. Hansen A/S (0.625 g/10 L) and CaCl₂) (1 g/10 L) were used. However, there are many more examples well known to the skilled person as alternative cultures that can be used in cheese making.

The composition for clotting milk used in Example 2 comprised:
a camel chymosin (CHY-MAX® Supreme)—compositions 5 and 6;
a camel chymosin (CHY-MAX® Supreme) and a non-encapsulated endothiapepsin (THERMOLASE®)—composition 7;
a camel chymosin (CHY-MAX® Supreme) and a non-encapsulated mucorpepsin (HANNILASE® XP)—composition 8.

CHY-MAX® M, THERMOLASE® and HANNILASE® XP are from Chr. Hansen A/S.

TABLE 4
Strength in IMCU/L of milk of each coagulant used in each composition
in Example 2. For each composition, two repeats were made.

| | Camel chymosin | Non-encapsulated | Non-encapsulated |
|---|---|---|---|
| Composition for | (CHY-MAX ® | endothiapepsin | mucurpepsin |
| clotting milk | Supreme) | (THERMOLASE ®) | (HANNILASE ® XP) |
| Composition 5 | 52.5 | 0 | 0 |
| Composition 6 | 40 | 0 | 0 |
| Composition 7 | 40 | 12.5 | 0 |
| Composition 8 | 40 | 0 | 12.5 |

The coagulant blends were realized 30 min before to be used. Alternatively, the coagulant blend can be made well in advance and be stored for at least 1 year prior to use.

The gels were cut at the same firmness, in particular 7.5 firmness index obtained with a CHYMOgraph® from Chr. Hansen A/S.

### Coagulation Kinetics

The dosage effect with CHY-MAX® Supreme, compositions 5 and 6, is very clear on coagulation profile. In particular, when composition 6 was used the firmness was obtained after 40 min and 30 s versus 31 min and 20 s when composition 5 was used.

Compositions having a strength of 52.5 IMCU/L of milk wherein 12.5 IMCU/L of milk corresponds to non-encapsulated endothiapepsin (composition 7) or a composition having 52.5 IMCU/L of milk wherein 12.5 IMCU/L of milk corresponds to non-encapsulated mucorpepsin (composition 8) share similar coagulation profiles between them. Further, their coagulation profiles are in between of the two CHY-MAX® Supreme curve (FIG. 4). In conclusion, the impact of mucorpepsin (HANNILASE® XP from Chr. Hansen) and endothiapepsin (THERMOLASE® from Chr. Hansen) on the coagulation profile is similar for both blends (or compositions) and the desired firmness was obtained after 35 min.

TABLE 5
Yield, whey composition, cheese composition and proteolysis
at 30 days, when compositions 5-8 are used for cheese-making.

| Composition for clotting milk | | | |
|---|---|---|---|
| 5 | 6 | 7 | 8 |

Yield

| | | | | |
|---|---|---|---|---|
| Cheese mass | 1120 | 1105 | 1110 | 1095 |
| Mass corrected | 1120.4 | 1096.8 | 1107.4 | 1082.5 |
| Yield | 11.20 | 10.97 | 11.07 | 10.82 |
| Relative yield | 2.16 | 0.00 | 0.97 | −1.30 |
| diff. vs CMS40 | | | | |
| in % | | | | |

Whey Composition

| | | | | |
|---|---|---|---|---|
| Fat | 0.57 | 0.65 | 0.56 | 0.6 |
| Protein | 0.93 | 0.96 | 0.94 | 0.94 |
| Lactose | 4.95 | 4.89 | 4.92 | 4.99 |
| DM | 7.25 | 7.34 | 7.22 | 7.28 |

Cheese Composition

| | | | | |
|---|---|---|---|---|
| Moisture | 44.98 | 45.41 | 45.13 | 45.63 |
| Fat | 26.85 | 26.52 | 26.76 | 26.48 |
| Fat/DM | 48.80 | 48.58 | 48.77 | 48.70 |
| Protein | 22.77 | 22.44 | 22.61 | 22.25 |
| Protein/DM | 41.38 | 41.11 | 41.21 | 40.92 |
| pH | 5.31 | 5.28 | 5.3 | 5.3 |

Proteolysis at 30 days

| | | | | |
|---|---|---|---|---|
| TN | 3.6003 | 3.5856 | 3.5991 | 3.6188 |
| SN | 0.2494 | 0.2758 | 0.5724 | 0.2824 |
| NPN | 0.152 | 0.1607 | 0.3555 | 0.1712 |
| SN/TN | 6.93 | 7.69 | 15.90 | 7.80 |
| NPN/TN | 4.22 | 4.48 | 9.88 | 4.73 |

The cheese composition was on target, in particular with a moisture of 45%, fat/DM of 48%, and the differences between cheeses were very small. In conclusion, the compositions used do not affect the cheese composition at this level. The same firmness at cutting allows to have the same cheese composition.

Cheese Yield Estimation from DM Recovery

Compositions 7 and 8 show that the first coagulant of the composition plays a role in cheese yield and that depending on which first coagulant is added to the mixture different yield results are obtained. For example, a first coagulant wherein said coagulant is a endothiapepsin (composition 7) surprisingly gives better yield results than when the first coagulant is a mucorpepsin (composition 8), even though the coagulation profiles were similar between compositions 7 and 8 (FIG. 4). In fact, composition 8 performs poorly when compared to compositions 5-7 and therefore is not a suitable candidate to obtain a combination of yield and flavor/texture suitable for the Cheddar market, for example.

### Proteolysis

The SN/TN after 1-month (30 days) of ripening shows the improvement of having a first coagulant in the composition, wherein the first coagulant is a endothiapepsin (composition 7), instead of a mucorpepsin (composition 8). Further, the secondary proteolysis (NPN/TN) is also affected by the type of blend used or more precisely by the value of the primary proteolysis (SN/TN) after 1-month of ripening. Thus, once again the effect of the first coagulant being endothiapepsin (such as THERMOLASE® from Chr. Hansen A/S or Suparen® from DSM) is herein demonstrated versus the effect of the first coagulant being a mucorpepsin.

### Casein Degradation

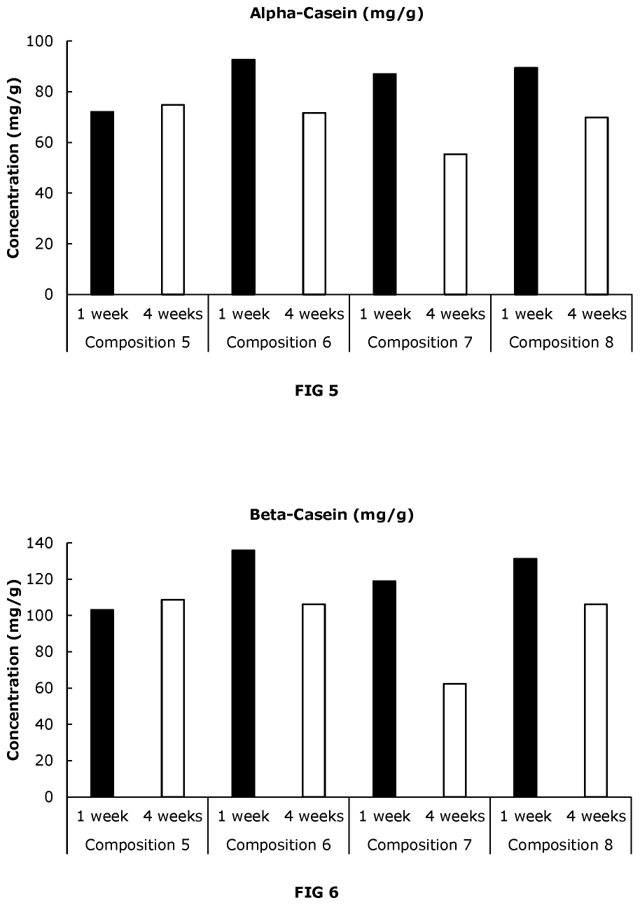

The casein degradation profile was analyzed for each composition, compositions 5-8 (FIG. 5-7) using electrophoresis (or LabChip® method). The casein degradation profiles correlated with the SN/TN results.

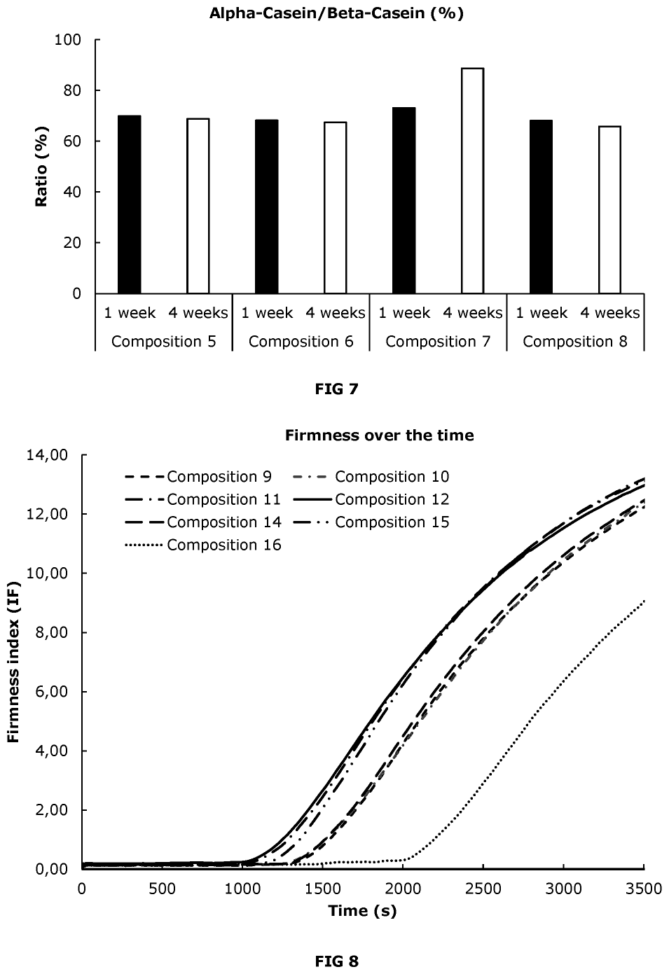

FIG. 7 shows that a composition comprising endothiapepsin (THERMOLASER) has a higher impact on the alpha-casein/beta-casein ratio, especially after 4-months of ripening. Thus, a small impact on the cheese texture but a higher impact on flavor formation can be obtained when using composition 7 in cheese making. In contrast, a composition comprising mucorpepsin (composition 8) does not change the alpha-casein/beta-casein ratio, similarly to compositions 5 and 6, which have one coagulant and not more than one.

In conclusion, Example 2 shows that the proteolysis pathway can be modulated with a composition comprising a camel chymosin and a endothiapepsin.

### Example 3

Continental cheeses were produced in the small cheese vat (10 L each) with a milk at Fat/protein ratio of 1. To produce them the DVS® culture C960 (0.625 g/10 L) from Chr. Hansen A/S+CaCl₂ (1 g/10 L) were used. However, there are many more examples well known to the skilled person as alternative cultures that can be used in cheese making.

Example 3 was focused on compositions comprising a camel chymosin (either CHY-MAX® M or CHY-MAX® Supreme) and an endothiapepsin (THERMOLASER) and where different ratios of both coagulants were tested (Table 6).

The composition for clotting milk used in Example 3 comprised:
a camel chymosin (CHY-MAX® M)—compositions 9 and 11;
a camel chymosin (CHY-MAX® M) and a non-encapsulated endothiapepsin (THERMOLASE®)—composition 10;
a camel chymosin (CHY-MAX® Supreme)—composition 12;
a camel chymosin (CHY-MAX® M) and a non-encapsulated endothiapepsin (THERMOLASE®)—compositions 13-16.

The compositions for clotting milk used in Example 3 are presented in Table 6. CHY-MAX® M, CHY-MAX® Supreme, and THERMOLASE® are from Chr. Hansen A/S.

TABLE 6
Strength in IMCU/L of milk of each coagulant used in each composition
in Example 3. For each composition, two repeats were made.

| Composition for | CHY-MAX ® Supreme | THERMOLASE ® |
|---|---|---|
| clotting milk | (IMCU/L of milk) | (IMCU/L of milk) |
| Composition 9 | 40 | 0 |
| Composition 10 | 40 | 12 |
| Composition 11 | 52 | 0 |
| Composition 12 | 52 | 0 |
| Composition 13 | 40 | 12 |
| Composition 14 | 46 | 6 |
| Composition 15 | 49 | 3 |
| Composition 16 | 34 | 6 |

The coagulant blends were realized 30 min before to be used. Alternatively, the coagulant blend can be made well in advance and be stored for at least 1 year prior to use.

The gels were cut at the same firmness (7.5 firmness index) with the CHYMOgraph® from Chr. Hansen A/S.

### Coagulation Kinetics

The coagulation kinetic shows 3 different profiles (FIG. 8):
Group 1 corresponds to composition 16, which was the slowest;
Group 2: corresponds to compositions 9, 10 and 14;
Group 3: corresponds to compositions 11, 12, 15, which were faster.

TABLE 7
Yield, whey composition, cheese composition and proteolysis at
30 days, when compositions 9-16 are used for cheese-making.

| | | | | | | | | |
|---|---|---|---|---|---|---|---|---|
| Composition | 9 | 10 | 11 | 12 | 13 | 14 | 15 | 16 |

Yield

| | | | | | | | | |
|---|---|---|---|---|---|---|---|---|
| Cheese | 1.1 | 1.095 | 1.105 | 1.125 | 1.105 | 1.1 | 1.11 | 1.085 |
| mass | | | | | | | | |
| Mass | 1.109 | 1.120 | 1.122 | 1.129 | 1.123 | 1.125 | 1.129 | 1.111 |
| corrected | | | | | | | | |
| Yield | 11.09 | 11.20 | 11.22 | 11.29 | 11.23 | 11.25 | 11.29 | 11.11 |
| Yield diff. | 0.0 | 1.0 | 1.2 | 1.8 | 1.2 | 1.5 | 1.7 | 0.2 |
| Vs CMM40 | | | | | | | | |

Proteolysis

| | | | | | | | | |
|---|---|---|---|---|---|---|---|---|
| TN | 3.6473 | 3.6835 | 3.6835 | 3.6289 | 3.672 | 3.7087 | 3.6953 | 3.6979 |
| SN | 0.3959 | 0.6704 | 0.4167 | 0.3147 | 0.6685 | 0.5763 | 0.4894 | 0.5739 |
| NPN | 0.2461 | 0.3969 | 0.2598 | 0.1838 | 0.4019 | 0.3407 | 0.2972 | 0.3412 |
| SN/TN | 10.85 | 18.20 | 11.31 | 8.67 | 18.21 | 15.54 | 13.24 | 15.52 |
| NPN/TN | 6.75 | 10.78 | 7.05 | 5.06 | 10.94 | 9.19 | 8.04 | 9.23 |

Whey Composition

| | | | | | | | | |
|---|---|---|---|---|---|---|---|---|
| Fat | 0.58 | 0.55 | 0.53 | 0.53 | 0.6 | 0.53 | 0.56 | 0.56 |
| protein | 0.97 | 0.97 | 0.94 | 0.93 | 0.96 | 0.95 | 0.96 | 0.95 |
| Lactose | 5.03 | 5 | 4.98 | 4.92 | 4.98 | 4.98 | 4.98 | 5.01 |
| DM | 7.33 | 7.27 | 7.21 | 7.15 | 7.29 | 7.19 | 7.24 | 7.23 |
| pH at whey | 6.58 | 6.59 | 6.58 | 6.58 | 6.59 | 6.58 | 6.58 | 6.58 |
| off | | | | | | | | |

Cheese Composition

| | | | | | | | | |
|---|---|---|---|---|---|---|---|---|
| Moisture | 44.54 | 43.74 | 44.15 | 44.81 | 44.11 | 43.73 | 44.08 | 43.69 |
| Fat | 26.88 | 27.40 | 27.12 | 26.67 | 27.06 | 27.41 | 27.15 | 27.28 |
| Fat/DM | 48.46 | 48.69 | 48.56 | 48.30 | 48.42 | 48.71 | 48.54 | 48.44 |
| Protein | 22.98 | 23.38 | 23.08 | 22.80 | 23.38 | 23.51 | 23.23 | 23.27 |
| Protein/DM | 41.44 | 41.54 | 41.32 | 41.31 | 41.82 | 41.78 | 41.53 | 41.32 |
| pH | 5.2 | 5.17 | 5.18 | 5.22 | 5.22 | 5.22 | 5.24 | 5.18 |

### Cheese and Whey Composition

Globally the cheese composition was on the target, in particular with a moisture of 44% and fat/DM of 48%; further the differences between cheeses are very small in the context of small cheese vats. Using the same firmness at cutting allows to have the same cheese composition. Therefore, it was possible to compare the different cheese parameters, namely proteolysis, yields, among other factors. On the whey, the impact of the coagulant blends, on fat, protein and total dry matter losses were observed.

Cheese Yield Estimation from DM Recovery

Example 3 shows that cheese yield and proteolysis can be surprisingly modulated by a composition of a first coagulant, wherein the first coagulant is an endothiapepsin and a second coagulant derived from or of camel. In particular, example 3 shows that in a composition of a camel chymosin and endothiapepsin, low dosages of endothiapepsin lead to increased cheese yield (compositions 13-15), at least upon to a level similar to a composition having only camel chymosin (composition 12), while simultaneously leading to an improvement in proteolysis versus the proteolysis of composition 12 (see Table 7) as it is further discussed below.

Proteolysis (after 1-Month of Ripening)

Example 3 shows that is possible to modulate the ST/TN according to the amount of endothiapepsin added, and that, in fact, the addition of endothiapepsin (THERMOLASER) to the composition, is a main ripening driver. For illustration, the compositions 14 (blends 46+6) and 16 (blends 34+6) give the same SN/TN level. The same applies to compositions 10 (blends 40+12) and 13 (blends 40+12).

Further, compositions 10 and 13-16 has a strong impact on the proteolysis (SN/TN and NPN/TN) after 1-month of ripening. In particular, these compositions have increasing dosages of endothiapepsin (6 to 12 IMCU/L of milk) and simultaneously show an increase SN/TN, from 13.24 to 18.21. For continental cheese, a value of 13% or more is an acceptable value for SN/TN. Furthermore, compositions 10 and 13-16 also acceptable yields.

Thus, example 3 demonstrates that it is possible to increase or maintain cheese yield with at the same time having the desired proteolysis.

### Casein Degradation

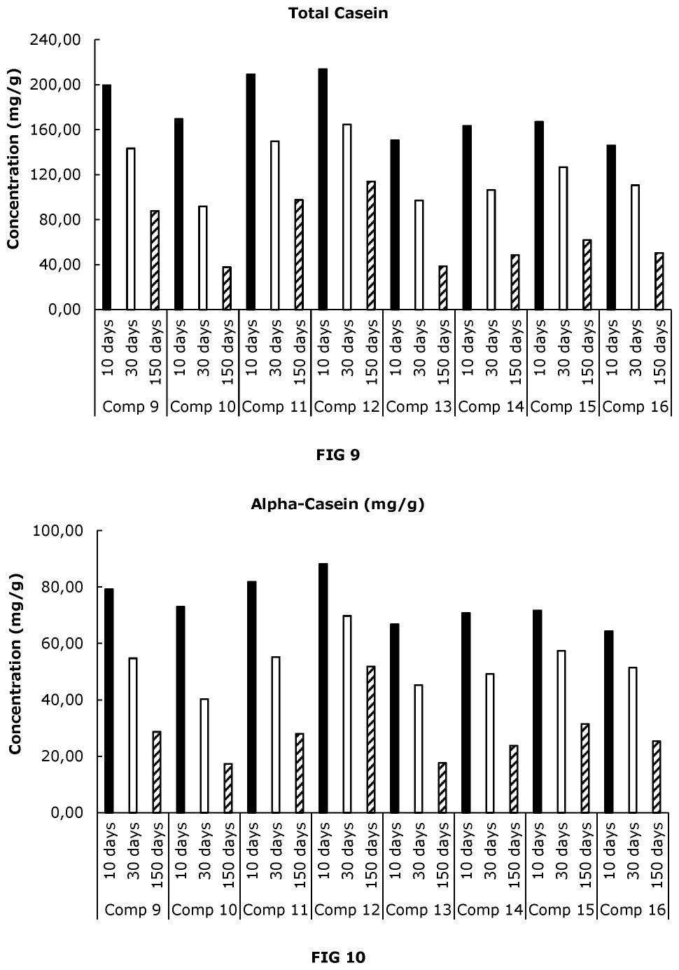

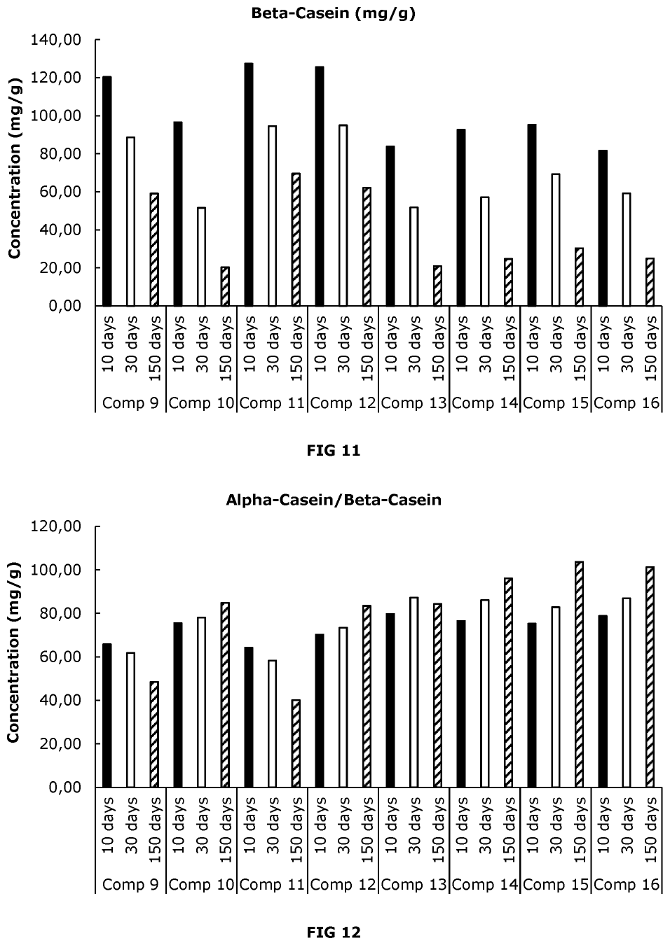

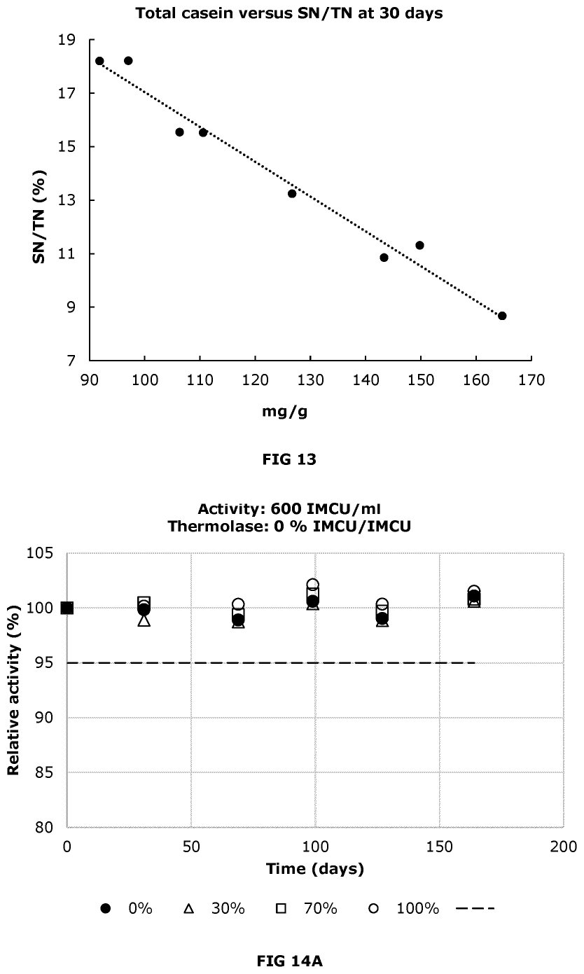

The casein degradation profile was analyzed for each composition, compositions 9-16 using electrophoresis (or LabChip® method). The casein degradation profiles correlated with the SN/TN results, as supported by FIG. 13. Total casein, alpha-casein, beta-casein and the ratio of alpha-casein/beta-casein was also determined for these samples at 10 days, 30 days (1M) and 150 days (4M) of ripening (FIG. 9-12).

The total casein depends on the coagulant blend used. Thus, FIG. 9 confirms the data of table 7 with regard to the proteolysis.

Further, from 10-days to 1-month the casein degradation followed the same trend for all compositions but that already after 10 days there is a difference between coagulants. The addition of an endothiapepsin, such as THERMOLASE® from Chr. Hansen, increases the proteolysis after 10-days versus 100% of a camel chymosin, such as CHY-MAX® M or Supreme both from Chr. Hansen, even with a small amount of endothiapepsin. These results confirm the fact that an endothiapepsin, and in particular THERMOLASE® from Chr. Hansen, leads the proteolysis while simultaneously promotes acceptable yields.

Additionally, different α-casein and β-casein degradation speed are obtained according to the coagulant type. The use of an endopeptidase (such as THERMOLASE® from Chr. Hansen) increases the β-casein degradation faster than the α-casein degradation, which means that the ratio α-casein/β-casein increases over the time when endopeptidase is used. This result is observed with all endopeptidase dosages tested (6 to 12 IMCU/L of milk).

In conclusion, the texture kinetic and the flavor formation can be modulated by the optimization of the ratio between a camel chymosin, e.g. CHY-MAX® M or CHY-MAX® Supreme both from Chr. Hansen, and endothiapepsin, e.g. THERMOLASE® from Chr. Hansen A/S or Suparen® from DSM. In particular, a ratio lower or close to 95% of a camel chymosin (CHY-MAX® M or CHY-MAX® Supreme, preferably CHY-MAX® Supreme) and 5% of endothiapepsin may be interesting.

### Example 4

This invention is also related to the development of a stability promoting formulation.

Blends of endothiapepsin (THERMOLASE® from Chr. Hansen A/S) and a camel chymosin (such as CHY-MAX® Supreme from Chr. Hansen A/S) were made at milk clotting activity 600 and 1000 IMCU/ml. Bulks of CHY-MAX® Supreme and THERMOLASE® where mixed to give desired ratio of the two, and subsequently blends were diluted to the desired milk clotting activity.

Blend ratio was expressed as percent THERMOLASE® activity of total activity. Samples were made having 0, 6, 12, 23 and 100% THERMOLASE® activity. Dilution of blends was done with 50% w/w glycerol, or with brine (12% NaCl pH 5.7), or with a mixture of the two (50% w/w glycerol and 12% NaCl pH 5.7).

After preparation, samples were aliquoted in glass vials, stored dark and under controlled temperature at 5° C. Milk clotting activity was determined every 30 days using the REMCAT method (as above described).

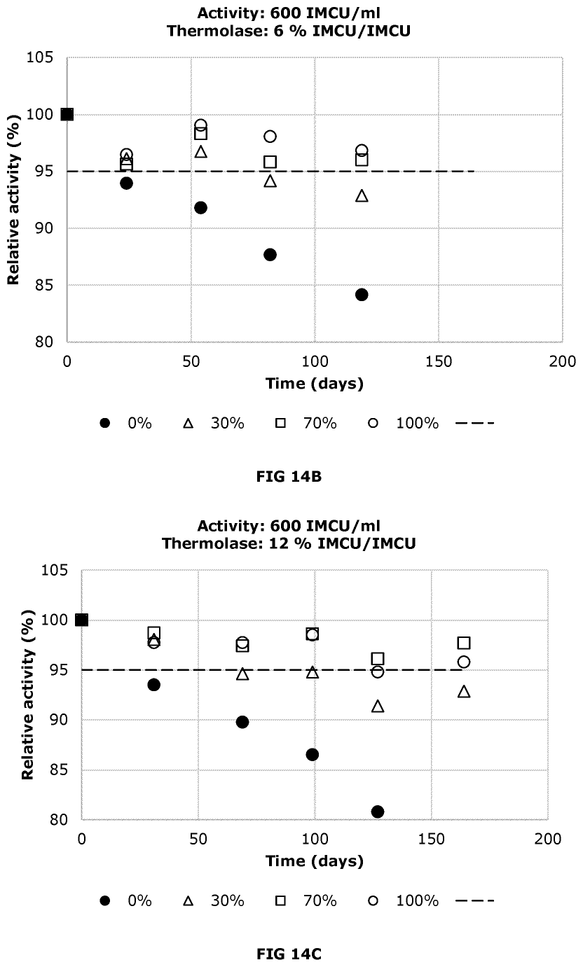

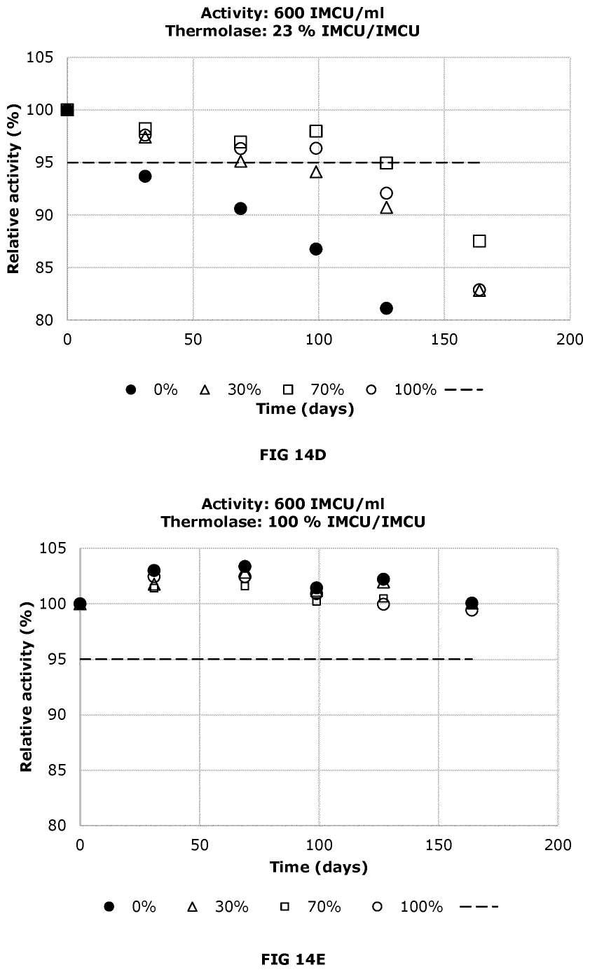

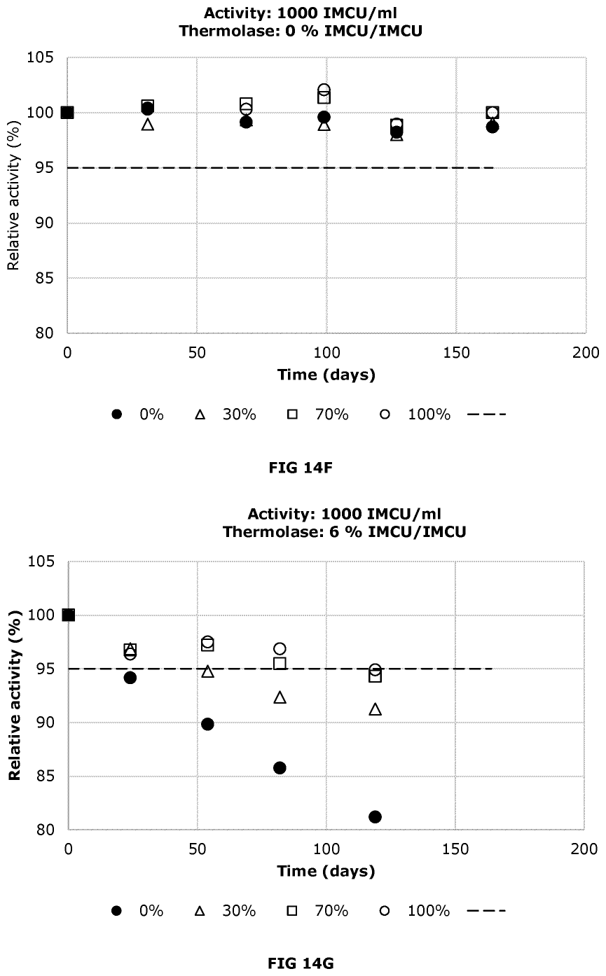

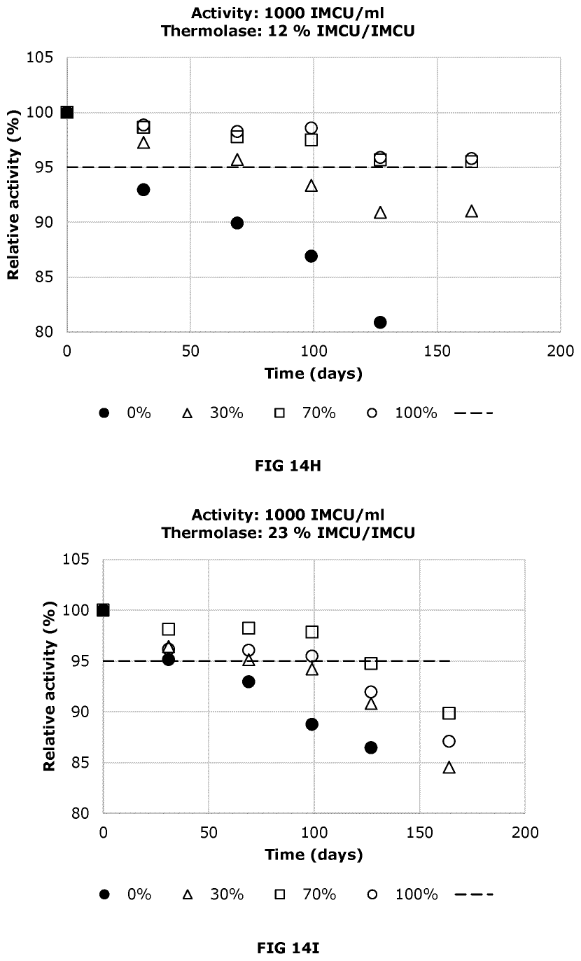

FIG. 14 shows the result of a formulation study where blends of THERMOLASE® and CHY-MAX® Supreme was made and diluted in different ways. In particular, blends of activity 600 and 1000 IMCU/ml were made with THERMOLASE® percent activity from 0 to 100, where 0 is pure CHY-MAX® Supreme and 100% is pure THERMOLASE®. Solution for blend dilution (% T dilution) varied from 0 to 100, where 0 is pure brine (12% NaCl) and 100% is pure 50% w/w glycerol. The time course of each sample shown in FIG. 14 was fitted to single exponential decay and a rate constant derived. The rate constant was used for extrapolating activity to 1 year in order to assess product stability.

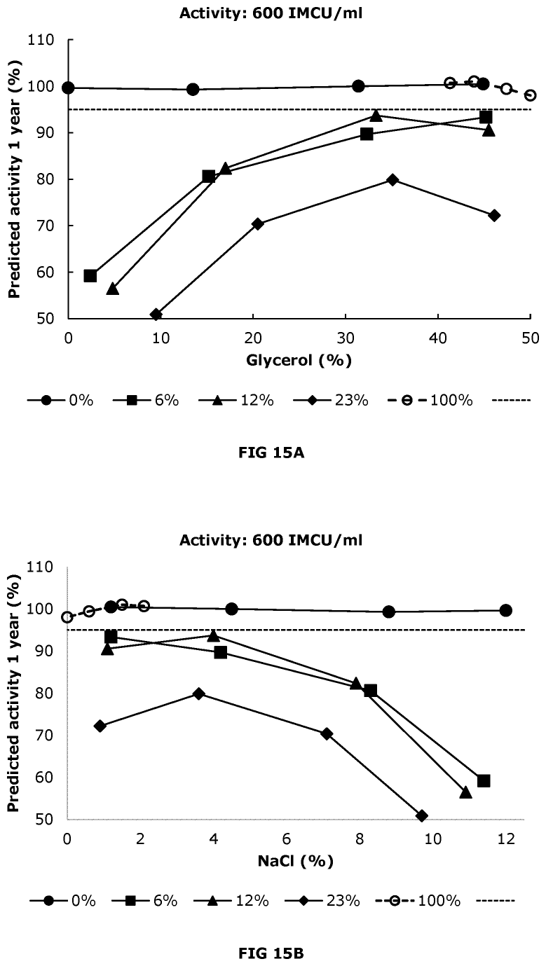

FIG. 15 (A and B) shows predicted activity after 1 year derived from time course of samples in FIG. 14. Concentration of glycerol and NaCl was calculated and plotted on x-axis in FIG. 15A and FIG. 15B, respectively. From the figure it is seen that pure THERMOLASE® was stable in all formulations covering 40-50% w/w glycerol and 0-2% NaCl. CHY-MAX® Supreme was stable in 1-12% NaCl and 0-45% w/w glycerol. However, stability of the blends with 6, 12 and 23% THERMOLASE® activity showed strong dependence on composition with increasing stability with increasing glycerol, and increasing stability with decreasing NaCl. None of the blends reached same stability as pure enzymes, and only two of the blend compositions approached desired stability of 95% or higher. Surprisingly, enzyme activity loss was the same irrespective of blend composition. The activity loss exceed content of THERMOLASE®. Thus, loss of enzyme activity was not due to one enzyme being less stable than the other.

In conclusion, FIG. 14 and FIG. 15 (A and B) show that stability of the blends of CHY-MAX® Supreme and THERMOLASE® was influenced by solvent composition despite stability of the pure enzymes was not. Stability of blends increased with increasing glycerol, and decreased with increasing NaCl.

### Example 5

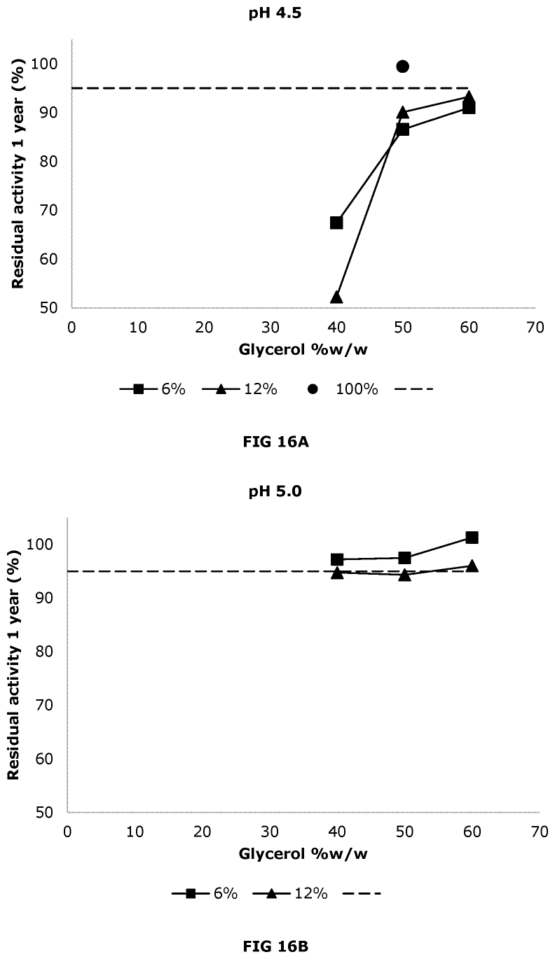

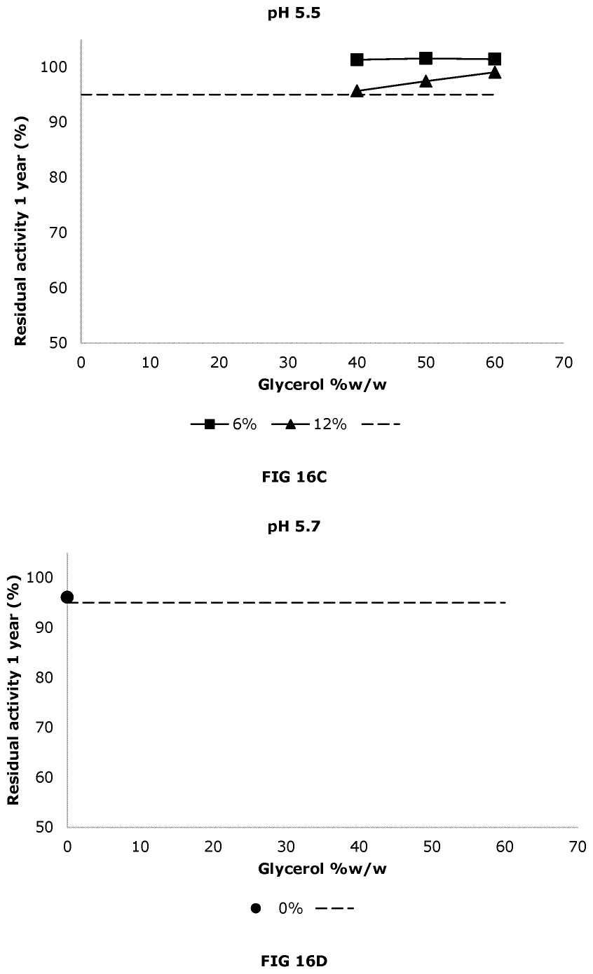

New samples were prepared to explore how increasing glycerol content and pH of blends influenced stability. Bulks of CHY-MAX® Supreme (12% w/v NaCl, pH 5.7) and THERMOLASE® (50% w/w glycerol, pH 4.5), both from Chr. Hansen A/S, were mixed in different ratios and diluted with 99% w/w glycerol and brine (12% NaCl, pH 5.7). Finally, pH was adjusted in samples by addition of either hydrochloric acid or sodium hydroxide. A factorial design was made with glycerol at levels 40, 50 and 60% w/w and pH at levels 4.5, 5.0 and 5.5. The result is shown in FIG. 16 showing relative activity extrapolated to 1 year of samples stored at 5° C. Blends with 6 and 12% THERMOLASE® retained close to 100% stability at 40-60% w/w glycerol, preferably with a pH of 5.0-5.5. At pH 4.5 there was an interaction between pH and glycerol with samples having 50-60% w/w glycerol being stable, whereas samples with pH 4.5 had significantly lower stability at 40% w/w glycerol.

### CONCLUSIONS

The present invention discloses that a composition comprising at least two coagulants, wherein one of the coagulants is a coagulant derived from or of Cryphonectria, such as Cryphonectria parasitica, in particular wherein the coagulant is an endothiapepsin having at least 80% sequence identity to SEQ ID Nos: 1 or 2, leads to the increase of yield in cheese curd while simultaneously maintaining proper levels of proteolysis for cheese making. Additionally, this invention shows that even a small addition of endothiapepsin significantly increases the proteolysis just a few days after cheese making and during the ripening but does not impact negatively the cheese yield.

A second coagulant of the composition may be a high C/P ratio coagulant such as a camel chymosin or a bovine chymosin. This invention shows that different chymosins, in particular different camel chymosins, give similar results.

Thus, this invention shows that to create a specific proteolysis pathway over time combined with adequate cheese yield it is important to select the right coagulant blend such as a blend of camel chymosin and endothiapepsin or a blend of bovine chymosin and endothiapepsin. In contrast, a blend of camel chymosin and mucorpepsin does not bring added value.

The cheese yield increase can be explained by higher fat partitioning into the cheese curd in the case of a mixture (compounding or blending) of coagulants. It is hypothesized that dosing of a chymosin and endothiapepsin, preferably the sequential dosing of a camel chymosin and endothiapepsin or of a bovine chymosin and endothiapepsin, leads to changes in cheese microstructure (casein network), which leads to increased retention of fat globules in the curd, as well as the loss of fine curd particles in to the whey is minimized.

Finally, the stability of the tested blends is influenced by the composition of the formulation. In particular, compositions (or blends) comprising 35-75% w/w of glycerol or 36-75% w/w of glycerol, preferably 40-60% w/w of glycerol or 50-60% w/w of glycerol, wherein % w/w indicates the weight of glycerol relative to total weight of the composition, eventually combined with a pH of 4.5-5.5, contribute to the stability of the compositions over time. In particular, compositions (or blends) having a pH of 4.5-5.5, preferably 5-5.5 or 4.5-5.0, eventually combined with 35-75% w/w of glycerol or 36-75% w/w of glycerol, preferably 40-60% w/w of glycerol or 50-60% w/w of glycerol, wherein % w/w indicates the weight of glycerol relative to total weight of the composition, contribute to the stability of the compositions over time.

### REFERENCES

WO2002036752; WO2016128476; WO2020229670; WO2013164479; WO2013164481; WO2016207214; WO2017198810; WO2017198829
Kim, S.-Y, Gunasekaran, S. and Olson, N. F., Combined use of chymosin and protease from Cryphonectria parasitica for control of meltability and firmness of Cheddar cheese, 2004, J. Dairy Sci. 87:274-283

1. A composition for clotting milk comprising
a first coagulant having at least 80% sequence identity with SEQ ID NO: 1 or 2; and
a second coagulant having at least 80% sequence identity with SEQ ID NO: 3 or 4 or 5 or 6 or 7.

2. The composition according to claim 1, wherein the first coagulant has at least 95% sequence identity with SEQ ID NO: 1 or 2.

3. The composition according to claim 1, wherein the first coagulant is an encapsulated endothiapepsin (EC 3.4.23.22).

4. The composition according to claim 1, wherein the second coagulant has at least 95% sequence identity with SEQ ID NO: 3 or 4 or 5 or 6 or 7.

5. The composition according to claim 1, comprising at most 25% of the first coagulant, based on the International Milk Clotting Units (IMCU)/ml of the first coagulant relative to the IMCU/ml of total coagulants in the composition.

6. The composition according to claim 1, comprising 1-25% of the first coagulant, based on the IMCU/ml of the first coagulant relative to the IMCU/ml of total coagulants in the composition.

7. The composition according to claim 1, comprising at least 75% of the second coagulant, based on the IMCU/ml of the second coagulant relative to the IMCU/ml of total coagulants in the composition.

8. The composition according to claim 1, comprising 75-99% of the second coagulant, based on the IMCU/ml of the second coagulant relative to the IMCU/ml of total coagulants in the composition.

9. The composition according to claim 1, further comprising a polyol selected from glycerol, sorbitol, monopropylene glycerol, sucrose, glucose, lactose and galactose.

10. The composition according to claim 1, comprising 25-65% w/w of the first coagulant and the second coagulant, relative to total weight of the composition.

11. The composition according to claim 1, wherein the composition has a pH of 4.5-5.5.

12. The composition according to claim 1, wherein the composition further comprises 40-60% w/w of glycerol relative to total weight of the composition and has a pH of 5-5.5.

13. A method for making a milk-based product comprising:
adding a first coagulant and a second coagulant to a milk base, wherein the first coagulant has at least 80% sequence identity with SEQ ID NO: 1 or 2 and the second coagulant has at least 80% sequence identity with SEQ ID NO: 3 or 4 or 5 or 6 or 7; and
processing the milk base to obtain the milk-based product.

14. The method according to claim 13, wherein the milk-based product is selected from Cheddar cheese, Continental cheese, and Swiss type cheese.

15. The method according to claim 13, wherein the milk-based product is a cheese and the method results in one or more of increased yield in cheese curd, increased yield in cheese, increased proteolysis after cheese making, and increased proteolysis during cheese ripening, as compared to an identical method carried out without the first coagulant.


SEQUENCE LISTING (52 sequences)

SEQ ID NO: 1 - protein, 419 aa, Cryphonectria parasitica
MSSPLKNALVTAMLAGGALSSPTKQHVGIPVNASPEVGPGKYSFKQVRNPNYKFNGPLSVKKTYLKYGVPIPAWLEDAVQNSTSGLAERSTGSATTTPIDSLDDAYITPVQIGTPAQTLNLDFDTGSSDLWVFSSETTASEVDGQTIYTPSKSTTAKLLSGATWSISYGDGSSSSGDVYTDTVSVGGLTVTGQAVESAKKVSSSFTEDSTIDGLLGLAFSTLNTVSPTQQKTFFDNAKASLDSPVFTADLGYHAPGTYNFGFIDTTAYTGSITYTAVSTKQGFWEWTSTGYAVGSGTFKSTSIDGIADTGTTLLYLPATVVSAYWAQVSGAKSSSSVGGYVFPCSATLPSFTFGVGSARIVIPGDYIDFGPISTGSSSCFGGIQSSAGIGINIFGDVALKAAFVVFNGATTPTLGFASK

SEQ ID NO: 2 - protein, 330 aa, Cryphonectria parasitica
STGSATTTPIDSLDDAYITPVQIGTPAQTLNLDFDTGSSDLWVFSSETTASEVDGQTIYTPSKSTTAKLLSGATWSISYGDGSSSSGDVYTDTVSVGGLTVTGQAVESAKKVSSSFTEDSTIDGLLGLAFSTLNTVSPTQQKTFFDNAKASLDSPVFTADLGYHAPGTYNFGFIDTTAYTGSITYTAVSTKQGFWEWTSTGYAVGSGTFKSTSIDGIADTGTTLLYLPATVVSAYWAQVSGAKSSSSVGGYVFPCSATLPSFTFGVGSARIVIPGDYIDFGPISTGSSSCFGGIQSSAGIGINIFGDVALKAAFVVFNGATTPTLGFASK

SEQ ID NO: 3 - protein, 381 aa, Camelus dromedarius
MRCLVVLLAALALSQASGITRIPLHKGKTLRKALKERGLLEDFLQRQQYAVSSKYSSLGKVAREPLTSYLDSQYFGKIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVLFGPSSDILKIQMAIGATENRYGEFDVNCGNLRSMPTVVFEINGRDYPLSPSAYTSKDQGFCTSGFQGDNNSELWILGDVFIREYYSVFDRANNRVGLAKAI

SEQ ID NO: 4 - protein, 323 aa, Camelus dromedarius [sequence omitted]

SEQ ID NO: 5 - protein, 381 aa, Camelus bactrianus
MRCLVVLLAALALSQASGITRIPLHKGKTLRKALKERGLLEDFLQRQQYAVSSKYSSLGKVAREPLTSYLDSQYFGKIYIGTPPQEFTVVFDTGSSDLWVPSIYCKSNACKNHHRFDPRKSSTFRNLGKPLSIHYGTGSIEGFLGYDTVTVSNIVDPNQTVGLSTEQPGEVFTYSEFDGILGLAYPSLASEYSVPVFDNMMDRHLVARDLFSVYMDRNGQGSMLTLGATDPSYYTGSLHWVPVTVQQYWQVTVDSVTINGVAVACVGGCQAILDTGTSVLFGPSSDILKIQMAIGATENRYGEFDVNCGSLRSMPTVVFEINGRDFPLAPSAYTSKDQGFCTSGFQGDNNSELWILGDVFIREYYSVFDRANNRVGLAKAI

SEQ ID NO: 6 - protein, 381 aa, Bos taurus
MRCLVVLLAVFALSQGAEITRIPLYKGKSLRKALKEHGLLEDFLQKQQYGISSKYSGFGEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSDILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 7 - protein, 323 aa, Bos taurus
GEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSDILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 8 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVIFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 9 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVIFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 10 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVIFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 11 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVIFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 12 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMVTLGAIDPSYYTGSLHWVPVTLQQYWQFTVDSVTINGVAVACVGGCQAILDTGTSVVFGPSSDILKIQMAIGATENEYGEFDVECDNIRSMPTVVFEINGRDYPLSPSAYTSKDQGFCTSGFQGDNNSELWILGDVFIREYYSVFDRANNRVGLAKAI

SEQ ID NO: 13 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVLFGPSSDILKIQMAIGATENEYGEFDVNCGNLRSMPTVVFEINGRDYPLSPSAYTSKDQGFCTSGFQGDNNSELWILGDVFIREYYSVFDRANNRVGLAKAI

SEQ ID NO: 14 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 15 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVLFGPSSDILKIQMAIGATENEYGEFDVECDNIRSMPTVVFEINGRDYPLSPSAYTSKDQGFCTSGFQGDNNSELWILGDVFIREYYSVFDRANNRVGLAKAI

SEQ ID NO: 16 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECGNLRSMPTVVFEINGRDYPLSPSAYTSKDQGFCTSGFQGDNNSELWILGDVFIREYYSVFDRANNRVGLAKAI

SEQ ID NO: 17 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVECDNIRSMPTVVFEINGRDYPLSPSAYTSKDQGFCTSGFQGDNNSELWILGDVFIREYYSVFDRANNRVGLAKAI

SEQ ID NO: 18 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGSMVTLGAIDPSYYTGSLHWVPVTLQQYWQFTVDSVTINGVAVACVGGCQAILDTGTSVIFGPSSDILKIQMAIGATENEYGEFDVECDNIRSMPTVVFEINGRDYPLSPSAYTSKDQGFCTSGFQGDNNSELWILGDVFIREYYSVFDRANNRVGLAKAI

SEQ ID NO: 19 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVIFGPSSDILKIQMAIGATENEYGEFDVECGNLRSMPTVVFEINGRDYPLSPSAYTSKDQGFCTSGFQGDNNSELWILGDVFIREYYSVFDRANNRVGLAKAI

SEQ ID NO: 20 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVIFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 21 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVLFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 22 - protein, 323 aa, Synthetic construct
GKVAREPLTSVLDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVNCDNIRSMPTVVFEINGRDYPLSPSAYTSKDQGFCTSGFQGDNNSELWILGDVFIREYYSVFDRANNRVGLAKAI

SEQ ID NO: 23 - protein, 323 aa, Synthetic construct
GKVAREPLTSVLDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVIFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 24 - protein, 323 aa, Synthetic construct
GKVAREPLTSILDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVNCDNIRSMPTVVFEINGRDYPLSPSAYTSKDQGFCTSGFQGDNNSELWILGDVFIREYYSVFDRANNRVGLAKAI

SEQ ID NO: 25 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVIFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 26 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 27 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECGNIRSMPTVVFEINGRDYPLSPSAYTSKDQGFCTSGFQGDNNSELWILGDVFIREYYSVFDRANNRVGLAKAI

SEQ ID NO: 28 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 29 - protein, 323 aa, Synthetic construct
GKVAREPLTSILDSQYFGTIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVLFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 30 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 31 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 32 - protein, 323 aa, Synthetic construct
GKVAREPLTSILDSQYFGTIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 33 - protein, 323 aa, Synthetic construct
GKVAREPLTSVLDSQYFGTIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 34 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 35 - protein, 323 aa, Synthetic construct
GKVAREPLTSVLDSQYFGTIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 36 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 37 - protein, 323 aa, Synthetic construct
GKVAREPLTSILDSQYFGKIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 38 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVVFGPSSDILKIQMAIGATENEYGEFDVECGNLRSMPTVVFEINGRDYPLSPSAYTSKDQGFCTSGFQGDNNSELWILGDVFIREYYSVFDRANNRVGLAKAI

SEQ ID NO: 39 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 40 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVIFGPSSDILKIQMAIGATENEYGEFDVNCGNLRSMPTVVFEINGRDYPLSPSAYTSKDQGFCTSGFQGDNNSELWILGDVFIREYYSVFDRANNRVGLAKAI

SEQ ID NO: 41 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVLFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 42 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 43 - protein, 323 aa, Synthetic construct
GKVAREPLTSILDSQYFGKIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 44 - protein, 323 aa, Synthetic construct
GKVAREPLTSILDSQYFGKIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVIFGPSSDILKIQMAIGATENEYGEFDVNCGNLRSMPTVVFEINGRDYPLSPSAYTSKDQGFCTSGFQGDNNSELWILGDVFIREYYSVFDRANNRVGLAKAI

SEQ ID NO: 45 - protein, 323 aa, Synthetic construct
GKVAREPLTSVLDSQYFGTIYIGTPPQEFTVVFDTGSSDLWVPSIYCKSNVCKNHHRFDPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMVTLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 46 - protein, 323 aa, Synthetic construct
GKVAREPLTSILDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVIFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 47 - protein, 323 aa, Synthetic construct
GKVAREPLTSILDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 48 - protein, 323 aa, Synthetic construct
GKVAREPLTSILDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMLTLGAIDPSYYTGSLHWVPVTLQQYWQFTVDSVTINGVAVACVGGCQAILDTGTSVIFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 49 - protein, 323 aa, Synthetic construct
GKVAREPLTSILDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 50 - protein, 323 aa, Synthetic construct
GKVAREPLTSVLDSQYFGTIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVVFGPSSDILKIQMAIGATENEYGEFDVNCDNLRSMPTVVFEINGRDYPLSPSAYTSKDQGFCTSGFQGDNNSELWILGDVFIREYYSVFDRANNRVGLAKAI

SEQ ID NO: 51 - protein, 323 aa, Synthetic construct
GKVAREPLTSILDSQYFGTIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGGMITLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI

SEQ ID NO: 52 - protein, 323 aa, Synthetic construct
GKVAREPLTSILDSQYFGKIYIGTPPQEFTVVFDTGSSDLWVPSIYCKSNVCKNHHRFNPRKSSTFRNLGKPLSIHYGTGSMEGFLGYDTVTVSNLVDPNQTVGLSTEQPGEVFTYSEFDGILGLAYPSLASEYSVPVFDNMMDRHLVARDLFSVYMDRNGQGGMVTLGAIDPSYYTGSLHWVPVTLQQYWQFTVDSVTINGVAVACVGGCQAILDTGTSVVFGPSSDILKIQMAIGATENEYGEFDVECDNLRSMPTVVFEINGRDYPLSPSAYTSKDQGFCTSGFQGDNNSELWILGDVFIREYYSVFDRANNRVGLAKAI